(19) United States
(12) Patent Application Publication
(10) Pub. No.: US 2014/0121185 A1
(43) Pub. Date: May 1, 2014

(54) COMPOUNDS AND COMPOSITIONS FOR USE IN THE TREATMENT AND PREVENTION OF CANCER AND PRECANCEROUS CONDITIONS, INFLAMMATION-RELATED DISORDERS, PAIN AND FEVER

(71) Applicant: Medicon Pharmaceuticals, Inc., Setauket, NY (US)

(72) Inventor: Basil RIGAS, Setauket, NY (US)

(73) Assignee: MEDICON PHARMACEUTICALS, INC., Setauket, NY (US)

(21) Appl. No.: 14/033,976

(22) Filed: Sep. 23, 2013

Related U.S. Application Data
(60) Provisional application No. 61/704,021, filed on Sep. 21, 2012.

(51) Int. Cl. C07F 9/6561; A61K 31/6615; C07F 9/12; A61K 45/06; C07F 9/09; A61K 31/675; A61K 31/661

(52) U.S. Cl. CPC C07F 9/6561; A61K 31/675; A61K 31/6615; A61K 31/661; A61K 45/06; C07F 9/094; C07F 9/12
USPC 514/81; 544/258; 558/160; 514/107; 558/179; 514/124; 558/159; 558/170; 514/119; 558/166

(57) ABSTRACT

Novel compounds and pharmaceutical compositions thereof for the treatment and/or prevention of cancer and precancerous conditions, inflammation-related disorders, pain and fever.

### FIELD OF THE INVENTION

The invention is directed to compounds and pharmaceutical compositions for the treatment and/or prevention of cancer and precancerous conditions, inflammation-related disorders, pain and fever.

### BACKGROUND OF THE INVENTION

Cancer remains the major cause of mortality in the industrial world. Despite significant advances in the early detection and treatment, the management of several widespread types of cancer, e.g. lung and pancreatic cancer remains difficult and patient survival is dismal. Widespread metastases often render surgery ineffectual leaving chemotherapy as the treatment of choice.

Inflammation is a common theme in cancer pathogenesis that has emerged in recent years. This seminal observation has provided the basis for our understanding of the chemopreventive properties of conventional anti-inflammatory drugs, most notably the so-called nonsteroidal anti-inflammatory drugs (NSAIDs). Their anticancer properties have been established primarily through epidemiological studied and more recently through a small number of interventional trials. For example, a recent meta-analysis of 91 epidemiological studies showed a significant exponential decline with increasing intake of NSAIDs in the risk for 7-10 malignancies including the four major types: colon, breast, lung, and prostate cancer (R. E. Harris et al., Oncology Reports 2005, 13, 559-83; T. L. Ratliff, J Urology 2005, 174, 787-8). All NSAIDs have anticancer effects restricted to cancer prevention (chemoprevention) (J. A. Baron, J Natl Cancer Inst 2004, 96, 4-5; E. J. Jacobs et al. J Natl Cancer Inst 2005, 97, 975-80; M. J. Thun et al., Novartis Foundation Symposium 2004, 256, 6-21; discussion 2-8, 49-52, 266-9).

However, the application of NSAIDs to cancer prevention is hampered by two serious limitations: (i) their suboptimal efficacy, which is less than 50% (K. Kashfi, B. Rigas, Biochem Soc Trans 2005, 33, (Pt 4), 724-727); and (ii) their side effects, which makes their long-term use problematic. For example, significant side effects are seen with sulindac, including gastrointestinal (up to 20% of the patients), CNS (up to 10%), skin rash and pruritus (5%), and transient elevations of hepatic enzymes in plasma (J. II Roberts, J. Morrow (2001), in: J. G. Hardman, L. E. Limbird (eds). Goodman and Gilman's The Pharmacological Basis of Therapeutics, 10th edn. McGraw-Hill: New York, pp. 687-731).

These observations suggest that a) the intrinsic anticancer properties of NSAIDs need enhancement if they are to be used successfully in the prevention and/or treatment of various types of cancer and b) their considerable side effects need be reduced, in particular if a chemoprevention application is contemplated, as it requires their long-term administration to subjects at risk of cancer.

There have been two main approaches to address these limitations. First, several groups have tried to chemically modify NSAIDs to enhance their efficacy and/or safety. Nitric oxide-donating NSAIDs and the amide derivative of sulindac represent two such approaches (B. Rigas Curr Opin Gastroenterol 2007, 23, 55-59; B. Rigas, J. L. Williams Nitric Oxide, 2008, 19, 199-204; G. A. Piazza et al. Cancer Prev Res (Phila) 2009, 2, 572-580). Second, there have been efforts to develop combination strategies that include conventional NSAIDs. The most successful combination includes sulindac and difluoromethylornithine (DFMO), which reduced by 69% the recurrence of adenomas in humans at risk for sporadic colon adenomas (F. L. Meyskens et al. Cancer Prev Res (Phila) 2008, 1, 9-11). DFMO inhibits ornithine decarboxylase, which catalyses the rate-limiting step in polyamine synthesis, whereas sulindac stimulates polyamine acetylation and export from the cell. This results in reduced intracellular polyamine levels leading to suppressed growth of cancer cells (E. W. Gerner, F. L. Meyskens Jr Nat Rev Cancer 2004, 4, 781-792; E. W. Gerner et al. Amino Acids 2007, 33, 189-195).

Aspirin, a derivative of salicylic acid invented over a century ago, remains the prototypical NSAID. The anti-inflammatory properties of aspirin are firmly established and are explained in part by its ability to inhibit the enzyme cyclooxygenase. The anticancer properties of aspirin are also known but are restricted to cancer prevention, an effect suspected to be mediated through multiple mechanistic effects.

WO 2009/023631 discloses compounds suitable for the treatment of inflammation and/or cancer. However, this reference does not address the treatment of pre-cancerous conditions and does not teach treatment of conditions such as pain or fever.

Thus, there is a need to develop compounds with improved efficacy and safety profiles for the treatment and/or prevention of various types of cancer and precancerous conditions as well as for the treatment of inflammation-related disorders, pain and fever. Given the prototypical role of aspirin in the control of inflammation, pain and fever, the inventor of the present application has focused on salicylic acid and also on 2-mercaptobenzoic acid and anthranilic acid and found a series of their structural derivatives with enhanced efficacy and safety compared to their parent compounds including aspirin (acetylsalicylic acid).

### SUMMARY OF THE INVENTION

The present invention provides novel therapeutics including a novel group of salicylic, 2-mercaptobenzoic and anthranilic acid derivatives and methods of using them in the treatment and/or prevention of disorders such as cancer and precancerous conditions and for the treatment of inflammation, pain and fever.

The present invention provides compounds of general Formula I

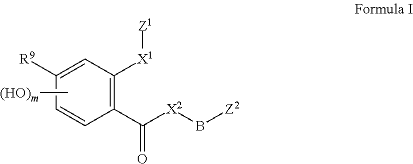

or an enantiomer, a diastereomer, a racemate, a tautomer, salt or hydrate thereof, wherein m=0 or 1;
X¹ and X² are independently selected from the group consisting of —O—, —S— and —NR′—, R¹ being hydrogen or C₁₋₆-alkyl;
B is an optionally substituted aliphatic, heteroaliphatic, aromatic, heteroaromatic or alkylaryl substituent having 1 to 40 carbon atoms;
Z¹ is selected from the group consisting of hydrogen, farnesyl and a folic acid residue;
Z² is selected from the group consisting of

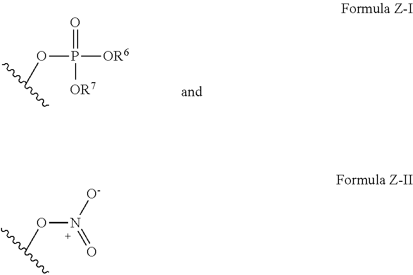

whereby Z² is preferably represented by Formula Z-I;
R⁶ being independently selected from hydrogen, C₁₋₁₀₀-alkyl and a polyethylene glycol residue,
R⁷ being independently selected from hydrogen, C₁₋₁₀₀-alkyl and a polyethylene glycol residue;
or B together with Z² forms a structure

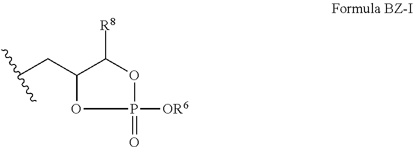

R⁶ being defined as above, and
R⁸ being independently selected from hydrogen, an aliphatic substituent with 1 to 22 carbon atoms, more preferred C₁₋₆-alkyl and a polyethylene glycol residue and
R⁹ being selected from hydrogen and trifluoromethyl.

A further aspect of the present invention relates to the compounds of Formula II

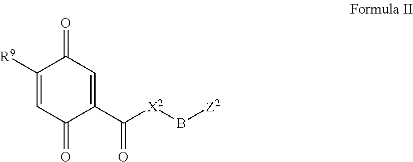

or an enantiomer, a diastereomer, a racemate, a tautomer, salt or hydrate thereof, wherein X², B, Z² and R⁹ are as defined above.

In one embodiment, the present invention relates to the compound of Formula I or Formula II for use in the treatment and/or prevention of cancer and precancerous conditions.

In a further embodiment, the present invention relates to the compound of Formula I or Formula II for use in the treatment and/or prevention of pain.

In yet another embodiment, the present invention relates to the compound of Formula I or Formula II for use in the treatment and/or prevention of inflammation-related diseases.

In yet another embodiment, the present invention relates to the compound of Formula I or Formula II for use as an antipyretic agent.

In a further aspect, the present invention is directed to a pharmaceutical composition comprising the compound of Formula I or Formula II, as described generally herein, and a pharmaceutically acceptable excipient. In a specific embodiment, the composition is useful for prevention and/or treatment or cancer or precancerous conditions, including but not limited to precancerous conditions such as benign prostatic hypertrophy, colon adenomas, actinic keratosis and various premalignant conditions of the lung, breast, oral cavity, cervix, and pancreas, and also cancer of the mouth, stomach, colon, rectum, lung, prostate, liver, breast, pancreas, skin, brain, head and neck, bones, ovaries, testicles, uterus, small bowel, lymphoma and leukemia.

Yet another embodiment of the present invention provides pharmaceutical compositions for the treatment of human and animal inflammation-related diseases, including, but not limited to neoplasms, rheumatologic diseases such as rheumatoid arthritis and Sjogren's syndrome; cardiovascular diseases, such as coronary artery disease, peripheral vascular disease and hypertension; neurodegenerative diseases, such as Alzheimer's disease and its variants or cerebrovascular diseases; and autoimmune diseases for example lupus erythematosus.

In addition to one or more compounds of Formula I or Formula II, the pharmaceutical compositions of the present invention can comprise one or more further pharmaceutical agents, for instance, compounds having anti-cancer activity. The compound of Formula I or Formula II can be administered alone or in combination with other active agents.

In yet another embodiment, the present invention provides methods for treating and/or prevention of cancer and precancerous conditions. The method typically comprises administering to a subject in need thereof a pharmaceutically effective amount of a compound of the present invention or of a pharmaceutical composition of the present invention.

In yet another aspect, the present invention provides methods for treating any disorder related to undesirable inflammation comprising administering to a subject (e.g. human patient or animal) in need thereof a therapeutically effective amount of a compound of Formula I or Formula II or a pharmaceutical composition comprising a compound of the invention. In a preferred embodiment, the disorder includes, but is not limited to rheumatologic diseases such as for example rheumatoid arthritis and Sjogren's syndrome; cardiovascular diseases, such as, for example, coronary artery disease, peripheral vascular disease and hypertension; neurodegenerative diseases, such as, for example, Alzheimer's disease and its variants or cerebrovascular diseases; autoimmune diseases such as for example lupus erythematosus; and other conditions characterized by chronic inflammation of organs such as for example the lung, such as chronic bronchitis or the sinuses, such as chronic sinusitis, and the skin, including eczema or atopic dermatitis, dryness of the skin and recurring skin rashes, contact dermatitis and seborrhoeic dermatitis, neurodermatitis and discoid and venous eczema.

In a further aspect, the invention is directed to a method for inhibiting inflammation, in particular, chronic inflammation in a subject in need thereof by administering to the subject an amount of the compound or composition of the present invention effective to inhibit inflammation. The subject may be a human patient or animal, for instance a mammal. Furthermore, the present invention is directed to a method for the treatment and/or prevention of cancer in a subject in need thereof by administering to the subject an amount of the compound or composition of the present invention.

In yet another aspect, the present invention provides methods for treating pain and/or fever. The invention further pertains to a method for alleviating pain, comprising administering to a subject in need thereof a pharmaceutically effective amount of a compound of the present invention or of a pharmaceutical composition of the present invention. The invention further pertains to a method for treating fever, comprising administering to a subject in need thereof a pharmaceutically effective amount of a compound of the present invention or of a pharmaceutical composition of the present invention.

The compounds represented by Formulae I and II may be used for the manufacture of pharmaceutical compositions for treatment of any diseases and disorders listed above.

### BRIEF DESCRIPTION OF THE FIGURES

FIG. 1. The growth of orthotopic MDA-MB231 human breast cancer xenografts treated with phospho-aspirin (PA) or acetylsalicylic acid (ASA), starting 1 wk prior to cell implantation.

FIG. 2. Phospho-aspirin (PA) metabolites in plasma and tumors and the effect of CYP isoforms.

FIG. 3. Time course of the levels of PA and its metabolites in PA-treated microsomes.

FIG. 4. P-FTS 35 inhibits pancreatic cancer cell growth in vitro in a concentration-dependent manner.

FIG. 5. P-FTS 35 inhibits the growth of pancreatic cancer xenografts. Left: inhibition of MIA PaCa-2 tumor growth in nude mouse by P-FTS 35.

FIG. 6. Growth of xenograft tumors treated with either vehicle (control) or P-FTS 35 and stained for Ki-67 expression (proliferation marker) or by the TUNEL method (apoptosis).

FIG. 7. P-FTS 35 inhibits Ras activation and reduces ERK1/2 and AKT activation in pancreatic cancer cells.

A: Immunoblots of Ras-GTP (active-Ras) and total K-Ras in Panc-1 cells treated without or with P-FTS 35, as indicated, for 24 h.

B: Immunoblots of Ras-GTP (active Ras) in cell protein extracts from Panc-1 cells treated with 50 μM P-FTS 35, or 50 μM FTS for 24 h. *P<0.05 vs. control.

C: Immunoblots of p-c-RAF, c-RAF, p-MEK, MEK, p-AKT and AKT in whole cell protein extracts from Panc-1 cells treated with P-FTS 35 as indicated. Loading control: β-actin.

FIG. 8. P-FTS 35 inhibits Ras activation and reduces ERK1/2 and AKT activation in pancreatic xenografts.

A: Immunoblots of Ras-GTP (active-Ras) and total K-Ras.

B: Immunoblots of p-ERK, ERK, p-AKT and AKT in whole cell protein extracts from MIA PaCa-2 xenografts. Loading control: β-actin.

C: Growth of xenograft tumors treated without or with P-FTS 35 and stained for p-ERK expression. The percentage of p-ERK positive cells/field were determined and expressed as the mean±SEM (*P<0.03).

FIG. 9. Phospho-valproic acid 64 enhances P-FTS-induced inhibition of pancreatic cancer cell growth. A: Cell viability was determined in MIA PaCa-2 after 24 h of incubation with P-V 64, P-FTS 35 or both. Results are expressed as % control. B: In this isobologram the additivity line connects the IC₅₀ value of each compound used alone. A and B represent two different dose pairs of each compound (their respective concentrations are shown in parentheses). The location of both A and B below the additivity line signifies synergy. C: The percentages of apoptotic cells determined by flow cytometry using the dual staining (Annexin V and PI). The percent of Annexin V(+) cells was calculated and results expressed as % control.

### DETAILED DESCRIPTION OF THE INVENTION

The term “aliphatic group”, as used herein, includes saturated or unsaturated, branched or unbranched aliphatic univalent or bivalent substituent groups. In the present application aliphatic group is intended to include, but is not limited to, alkyl, alkylene, alkenylene, alkynylene and alkadienylene substituent groups. According to the present invention, the aliphatic group has 1 to 100 carbon atoms, more preferred 1 to 42 carbon atoms, further preferred 1 to 22 carbon atoms, more preferred 1 to 15 carbon atoms, further preferred 1 to 10 carbon atoms, even more preferred 1 to 6 carbon atoms, for instance 4 carbon atoms. Most preferably, the aliphatic group is C₁₋₆-alkylene, e.g. methylene, ethylene, trimethylene and tetramethylene.

The term “alkyl” used is the present application relates a saturated branched or unbranched aliphatic univalent substituent group. Preferably, the alkyl group has 1 to 100 carbon atoms, more preferred 1 to 42 carbon atoms, even more preferred 1 to 42 carbon atoms, further preferred 1 to 22 carbon atoms, more preferred 1 to 6 carbon atoms, even more preferred 1 to 3 carbon atoms. Accordingly, examples of the alkyl group include methyl, ethyl, n-propyl, isopropyl, n-butyl, isobutyl, sec-butyl, tert-butyl, n-pentyl and n-hexyl and preferable examples include methyl, ethyl, n-propyl and isopropyl, whereby ethyl and isopropyl are particularly preferred.

The term “alkylene” used is the present application relates a saturated branched or unbranched aliphatic bivalent substituent group. Preferably, the alkylene group has 1 to 6 carbon atoms, more preferred 1 to 3 carbon atoms. Accordingly, examples of the alkylene group include methylene, ethylene, trimethylene, propylene, tetramethylene, isopropylidene, pentamethylene and hexamethylene. Preferable examples of the alkylene group include methylene, ethylene, trimethylene and tetramethylene, whereby tetramethylene is particularly preferred.

The term “alkenylene” as used is the present application is an unsaturated branched or unbranched aliphatic bivalent substituent group having a double bond between two adjacent carbon atoms. Preferably, the alkenylene group has 2 to 6 carbon atoms, more preferred 2 to 4 carbon atoms. Accordingly, examples of the alkenylene group include but are not limited to vinylene, 1-propenylene, 2-propenylene, methylvinylene, 1-butenylene, 2-butenylene, 3-butenylene, 2-methyl-1-propenylene, 2-methyl-2-propenylene, 2-pentenylene, 2-hexenylene. Preferable examples of the alkenylene group include vinylene, 1-propenylene and 2-propenylene, whereby vinylene is particularly preferred.

The term “alkynylene” as used is the present application is an unsaturated branched or unbranched aliphatic bivalent substituent group having a triple bond between two adjacent carbon atoms. Preferably, the alkynylene group has 2 to 6 carbon atoms, more preferred 2 to 4 carbon atoms. Examples of the alkynylene group include but are not limited to ethynylene, 1-propynylene, 1-butynylene, 2-butynylene, 1-pentynylene, 2-pentynylene, 3-pentynylene and 2-hexynylene. Preferable examples of the alkenylene group include ethynylene, 1-propynylene and 2-propynylene, whereby ethynylene is particularly preferred.

The term “alkadienylene” as used is the present application is an unsaturated branched or unbranched aliphatic bivalent substituent group having two double bonds between two adjacent carbon atoms. Preferably, the alkadienylene group has 4 to 10 carbon atoms. Accordingly, examples of the alkadienylene group include but are not limited to 2,4-pentadienylene, 2,4-hexadienylene, 4-methyl-2,4-pentadienylene, 2,4-heptadienylene, 2,6-heptadienylene, 3-methyl-2,4-hexadienylene, 2,6-octadienylene, 3-methyl-2,6-heptadienylene, 2-methyl-2,4-heptadienylene, 2,8-nonadienylene, 3-methyl-2,6-octadienylene, 2,6-decadienylene, 2,9-decadienylene and 3,7-dimethyl-2,6-octadienylene groups, whereby 2,4-pentadienylene is particularly preferred.

As used herein, “aromatic substituent” is intended to mean any stable monocyclic, bicyclic or polycyclic carbon ring of up to 10 atoms in each ring, wherein at least one ring is aromatic, and may be unsubstituted or substituted. Examples of such aromatic substituents include phenyl, p-toluenyl(4-methylphenyl), naphthyl, tetrahydronaphthyl, indanyl, biphenyl, phenanthryl, anthryl or acenaphthyl. In cases where the aromatic substituent is bicyclic and one ring is non-aromatic, it is understood that attachment is via the aromatic ring.

The term “arylalkyl substituents” refers to alkyl substituents as described above wherein one or more bonds to hydrogen contained therein are replaced by a bond to an aryl substituent as described above. It is understood that an arylalkyl substituents is connected to the carbonyl group if the compound of Formula I of Formula II through a bond from the alkyl substituent. Examples of arylalkyl substituents include, but are not limited to benzyl(phenylmethyl), p-trifluoromethylbenzyl(4-trifluoromethylphenylmethyl), 1-phenylethyl, 2-phenylethyl, 3-phenylpropyl, 2-phenylpropyl and the like.

The term “heteroaromatic substituent” as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic heteroaromatic substituents include phenyl, pyridine, pyrimidine or pyridizine rings that are
a) fused to a 6-membered aromatic (unsaturated) heterocyclic ring having one nitrogen atom;
b) fused to a 5- or 6-membered aromatic (unsaturated) heterocyclic ring having two nitrogen atoms;
c) fused to a 5-membered aromatic (unsaturated) heterocyclic ring having one nitrogen atom together with either one oxygen or one sulfur atom; or
d) fused to a 5-membered aromatic (unsaturated) heterocyclic ring having one heteroatom selected from O, N or S.

Heteroaromatic substituents within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The aliphatic, heteroaliphatic, aromatic and heteroaromatic substituents can be optionally substituted one or more times, the same way or differently with any one or more of the following substituents including, but not limited to: aliphatic, heteroaliphatic, aromatic and heteroaromatic substituents, aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent substituents taken together may represent a 4, 5, 6, or 7-membered substituted or unsubstituted alicyclic or heterocyclic substituents. Additional examples of generally applicable substituents are illustrated by the specific embodiments shown below.

The terms “halo” and “halogen” refer to a halogen atom selected from the group consisting of F, Cl, Br and I. Preferably the halogen atom is Cl or Br, whereby Cl is particularly preferred.

The term “halogenated alkyl substituent” refers to an alkyl substituents as defined above which is substituted with at least one halogen atom. In a preferred embodiment, the halogenated alkyl substituent is perhalogenated. In a more preferred embodiment, the halogenated alkyl substituent is a univalent perfluorated substituent of formula CₙF₂ₙ₊₁. Preferably, the halogenated alkyl substituent has 1 to 6 carbon atoms, even more preferred 1 to 3 carbon atoms. Accordingly, examples of the alkyl group include trifluoromethyl, 2,2,2-trifluoroethyl, n-perfluoropropyl, n-perfluorobutyl and n-perfluoropentyl. Preferable examples of halogenated alkyl substituents include trifluoromethyl and 2,2,2-trifluoroethyl, whereby trifluoromethyl is particularly preferred.

The term “polyethylene glycol” (PEG) refers to a compound of formula H—(OCH₂CH₂)ₙOH in which n has a value typically from 21 to 135, but not restricted to this range. Commercial polyethylene glycols having number average molecular weights of 1,000, 1,500, 1,540, 4,000 and 6,000 are useful in this invention. These solid polyethylene glycols have melting points of 35° C. to 62° C. and have boiling or flash points ranging from 430° C. to over 475° C. The preferred polyethylene glycol residues falling within the definition of the present invention are those having the formula —(OCH₂CH₂)ₙOCH₃ in which n is from 21 through 135, n preferably n from 40 to 50.

The present inventor found that the compounds of general Formula IV, whereby the substituents X¹, Z¹, B, R⁹, X² and Z² are as defined above, are capable of undergoing metabolism as depicted in Scheme 1. Furthermore, the inventor found that, contrary to the compounds of Formula IV, acetylsalicylic acid itself is not capable of undergoing these transformations in vivo.

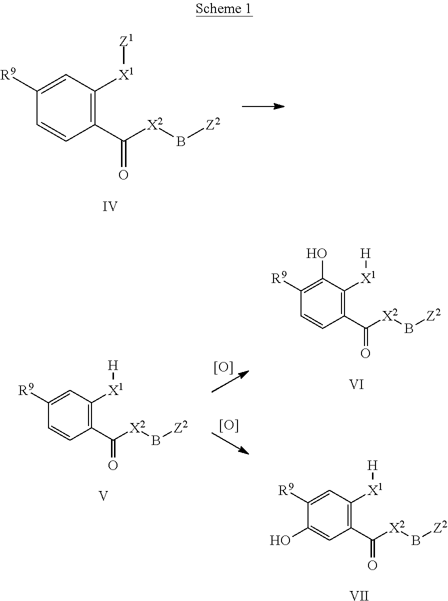

The present invention is inter alia based on a surprising finding that the metabolites represented by the general Formulae VI and VII have a pronounced anti-cancer activity and are therefore suitable for the treatment and/or prevention of cancer and precancerous conditions. Moreover, these compounds are capable of undergoing oxidation to highly reactive quinone-type derivatives, in particular to benzoquinones of Formula II (Scheme 2), the cytostatic properties of which are likely to explain the anti-cancer activity of the parent compounds of general Formula IV.

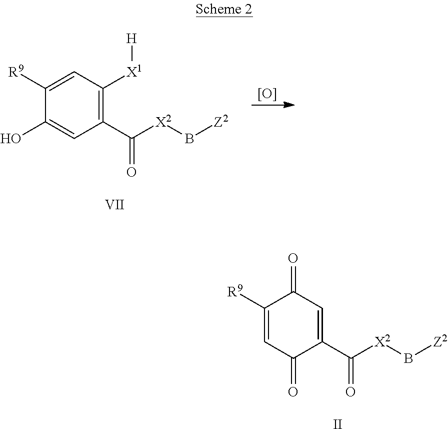

Accordingly, one aspect of the present invention relates to the compound of Formula I:

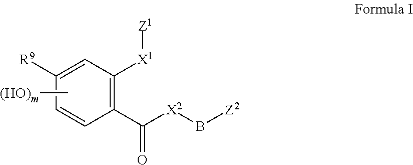

or an enantiomer, a diastereomer, a racemate, a tautomer, salt or hydrate thereof.

The number of hydroxy substituents m may be 0 or 1. When m is 1, the position of the hydroxy group in the aromatic moiety is not particularly limited. Thus, said hydroxy group may be located in the 2, 3, 4, 5 or 6 position relative to the carbonyl group, 5 being particularly preferred.

The substituents X¹ and X² in Formula I can be —O—, —S— or —NR¹—, R¹ being hydrogen or an alkyl group having 1 to 6 carbon atoms, more preferred 1 to 3 carbon atoms such as, for instance, methyl or ethyl, preferably methyl. The substituent R⁹ may be hydrogen or trifluoromethyl, hydrogen being particularly preferred.

In some preferred embodiments, Z¹ in Formula I is a folic acid residue. The folic acid residue may be selected from the residues defined by the Formulae Z-III, Z-IV or Z-V which are shown below:

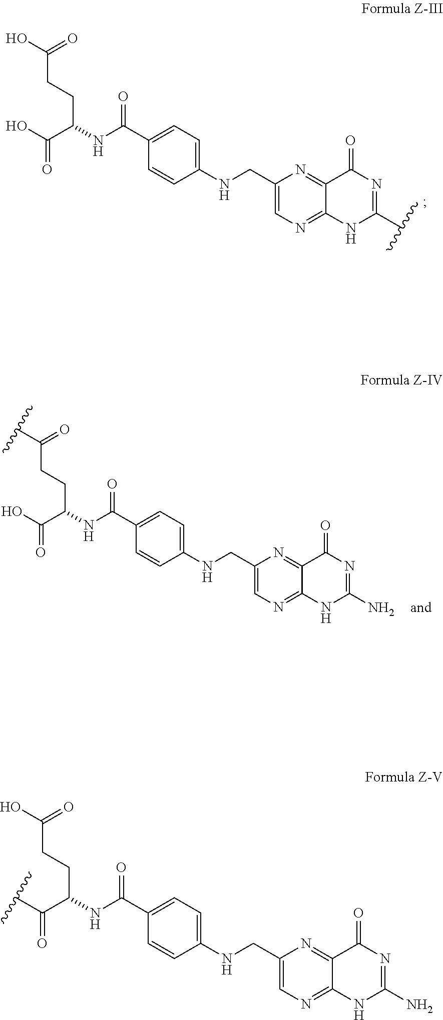 [SMILES: *c1nc(=O)c2nc(CNc3ccc(C(=O)N[C@@H](CCC(=O)O)C(=O)O)cc3)cnc2[nH]1 ; *C(=O)CC[C@H](NC(=O)c1ccc(NCc2cnc3[nH]c(N)nc(=O)c3n2)cc1)C(=O)O ; *C(=O)[C@H](CCC(=O)O)NC(=O)c1ccc(NCc2cnc3[nH]c(N)nc(=O)c3n2)cc1]

Without wishing to be bound by any theory it is believed that compounds of the present invention having Z¹ represented by Formulae Z-III to Z-V have a particularly strong anti-cancer activity. In particular, anti-cancer activity of these compounds is usually higher than the activity of the corresponding compounds in which Z¹ is hydrogen. In these embodiments, the substituent X¹ may, for instance, be selected from —O— and —NR¹—, whereby —O— and —NH— are particularly preferred.

In other preferred embodiments, the substituent X¹ in Formula I is —O— and the substituent Z¹ is hydrogen, the compound of the present invention being represented by Formula III:

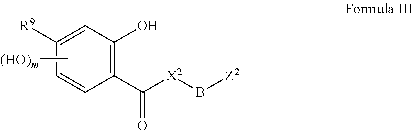

In yet other preferred embodiments, Z¹ in Formula I is farnesyl (IUPAC name: (2E,6E)-3,7,11-trimethyldodeca-2,6,10-trienyl) the structure of which is shown below:

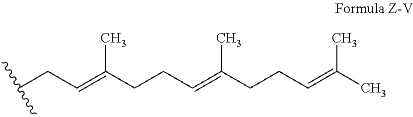 [SMILES: *C/C=C(\C)CC/C=C(\C)CCC=C(C)C]

In these embodiments, the substituent X¹ may be selected from —S—, —O— and —NH—, whereby —S— is particularly preferred.

The substituent Z² in Formula I may be represented by Formulae Z-I or Z-II

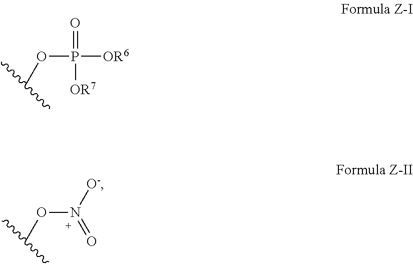

Formula Z-I being particularly preferable;

R⁶ and R⁷ being independently selected from hydrogen, C₁₋₁₀₀-alkyl and polyethylene glycol substituent. Preferably, the substituents R⁶ and R⁷ are independently selected from C₁₋₃-alkyl and (OCH₂CH₂)ₙOCH₃, whereby n is from 40 to 50, whereby it is particularly preferred that R⁶ and R⁷ are identical C₁₋₃-alkyl substituents, for instance ethyl.

The substituent B in Formula I is an optionally substituted aliphatic, heteroaliphatic, aromatic, heteroaromatic or alkylaryl substituent having 1 to 40 carbon atoms. In particular, the substituent B may be selected from the group consisting of

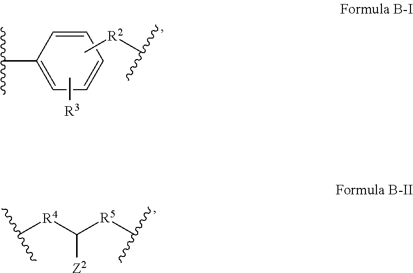

and an aliphatic substituent with 1 to 40, preferably with 1 to 22 carbon atoms.

Substituents R², R⁴, and R⁵ can be the same or different alkylene substituent having 1 to 3 carbon atoms. Preferably the substituent R² in Formula B-I is selected from the group consisting of methylene, ethylene and trimethylene; in a more preferred embodiment, R² is methylene or ethylene, whereby methylene is particularly preferred.

In yet another preferred embodiment, the substituent B is represented by Formula B-II and R⁴ and R⁵ are identical alkylene substituents having 1 to 3 carbon atoms. In a particularly preferred embodiment, R⁴ and R⁵ are both methylene substituents so that the substituent B in Formula I is represented by Formula B-IV:

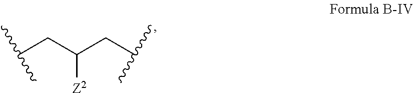

Z² being defined above.

In a particularly preferred embodiment, the substituent B together with X² and Z² forms a glycerol ester residue.

R³ in Formula B-I can be hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)₂NH—C₁₋₆-alkyl, cyano, halo or hydroxy substituents. In some preferred embodiments, R³ in Formula B-I is selected from hydrogen, an alkyl having 1 to 3 carbon atoms, halo and methoxy. Preferably, R³ is selected from the group consisting of hydrogen, methyl, fluoro, chloro, bromo and methoxy, preferably from the group consisting of hydrogen, methyl, chloro and fluoro. In a particularly preferred embodiment, R³ represents hydrogen so that the substituent B is represented by Formula B-III:

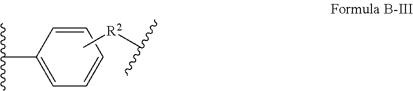

The substitution pattern of the substituent B in Formulae B-I and B-III is not particularly limited. When the substituent B is represented by Formula B-Ill, the aromatic moiety of the substituent B can be 1,2- or 1,3- or 1,4-substituted. Preferably, the aromatic moiety of B is 1,4-substituted so that B is represented by Formula B-V:

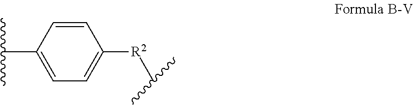

In yet another embodiment of the present invention, the substituent B is an aliphatic substituent, preferably having 1 to 40 carbon atoms, more preferred 1 to 22 carbon atoms, further preferred 1 to 6 carbon atoms. In a particularly preferred embodiment, B is selected from the group consisting of alkylene substituents with 1 to 6 carbon atoms, alkenylene substituent having 2 to 6 carbon atoms and alkynylene substituent having 2 to 6 carbon atoms. In a more preferred embodiment, the substituent B is an alkylene substituent with 1 to 6 carbon atoms, preferably an alkylene substituent with 1 to 4 carbon atoms, even more preferred tetramethylene substituent.

In a further embodiment of the present invention, the substituent B together with the substituent Z² forms a structure

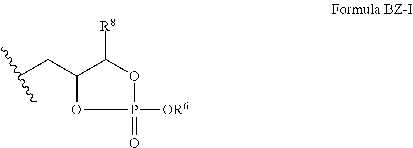

R⁶ being defined as above,
R⁸ being independently selected from hydrogen, an aliphatic substituent, preferably with 1 to 22 carbon atoms, more preferred C₁₋₆-alkyl, and a polyethylene glycol residue.

In yet another preferred embodiment, R⁸ is hydrogen and the substituent B together with the substituent Z² forms a structure

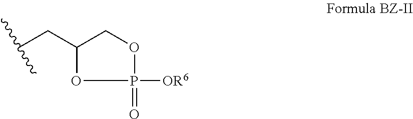

A further aspect of the present invention relates to a compound Formula II

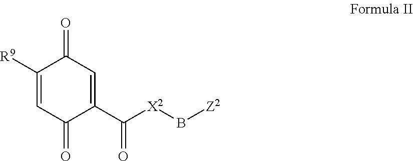

or an enantiomer, a diastereomer, a racemate, a tautomer, salt or hydrate thereof. The substituents X², B and Z² are as defined above.

In one embodiment of the invention, m is 0, R⁹ is hydrogen and the substituent Z¹ is a folic acid residue represented by Formulae Z-III to Z-V. Accordingly, the compounds of Formula I include but are not limited to compounds 1 to 18 the structures of which are shown below:

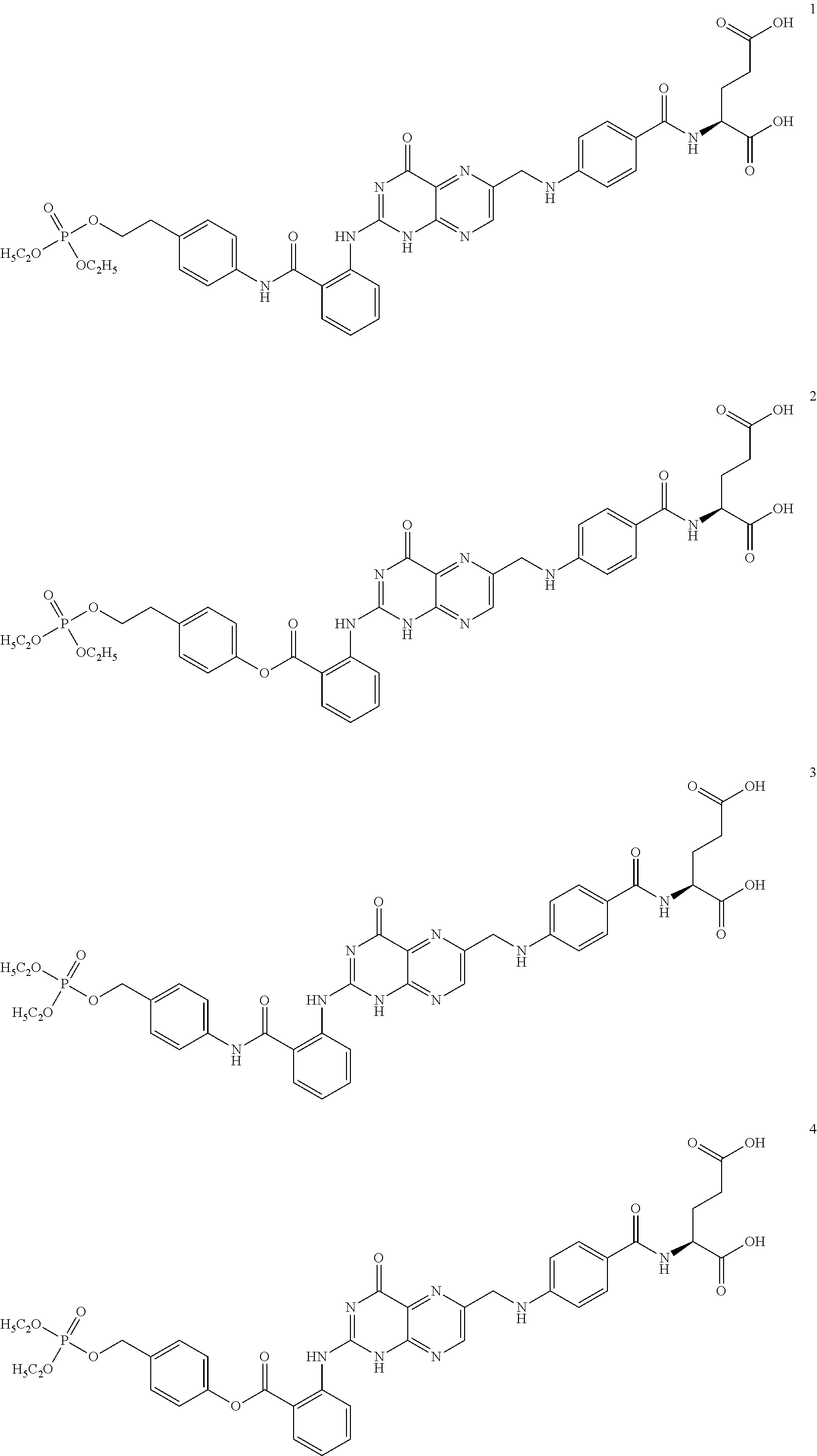

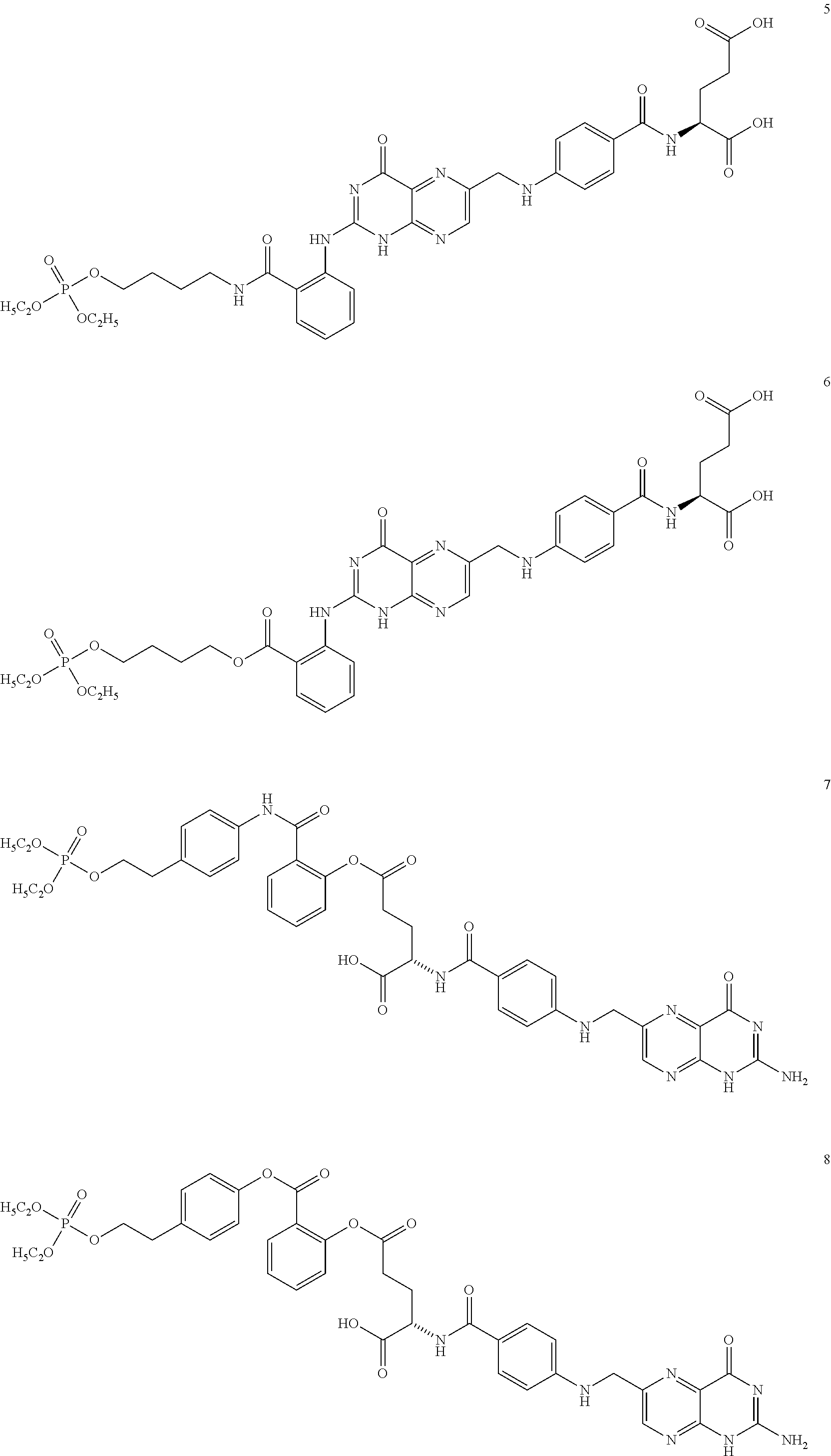

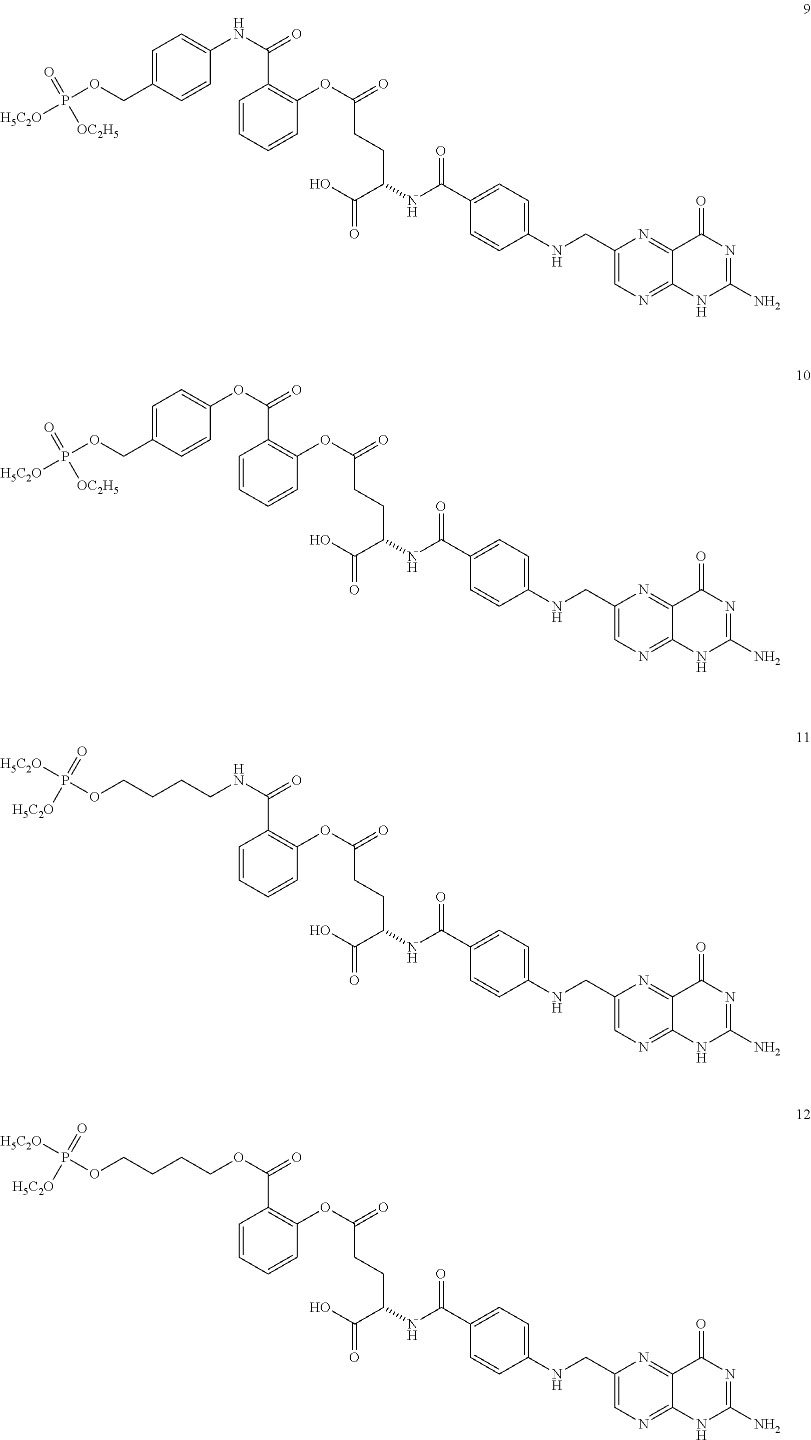

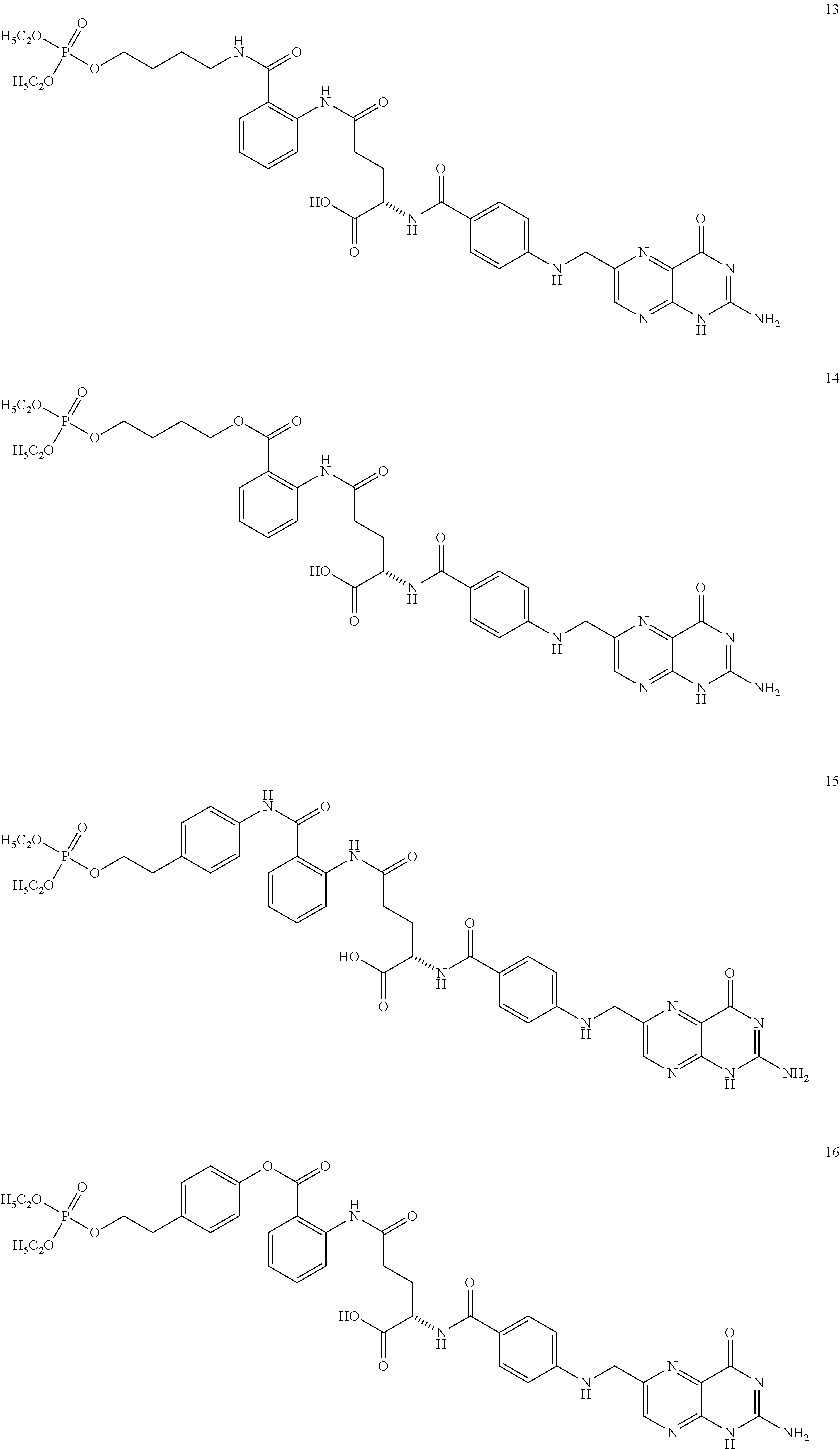

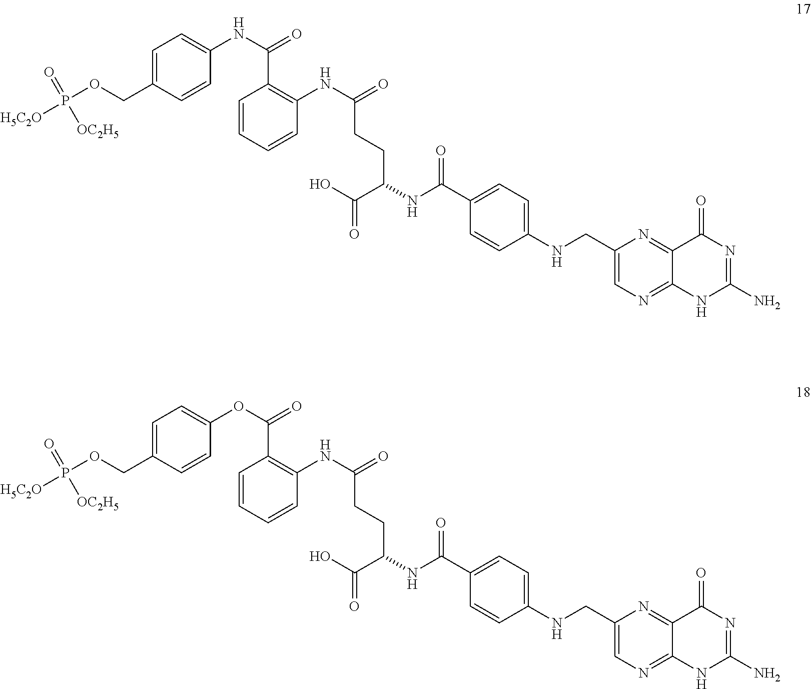

In another embodiment, m is 0, R⁹ is hydrogen and the substituent Z¹ is farnesyl. Without wishing to be bound by a theory, applicant believes that these compounds may be advantageously employed for the treatment of cancers such as pancreatic cancer. Accordingly, the compounds of Formula I include but are not limited to compounds 19 to 36 shown below:

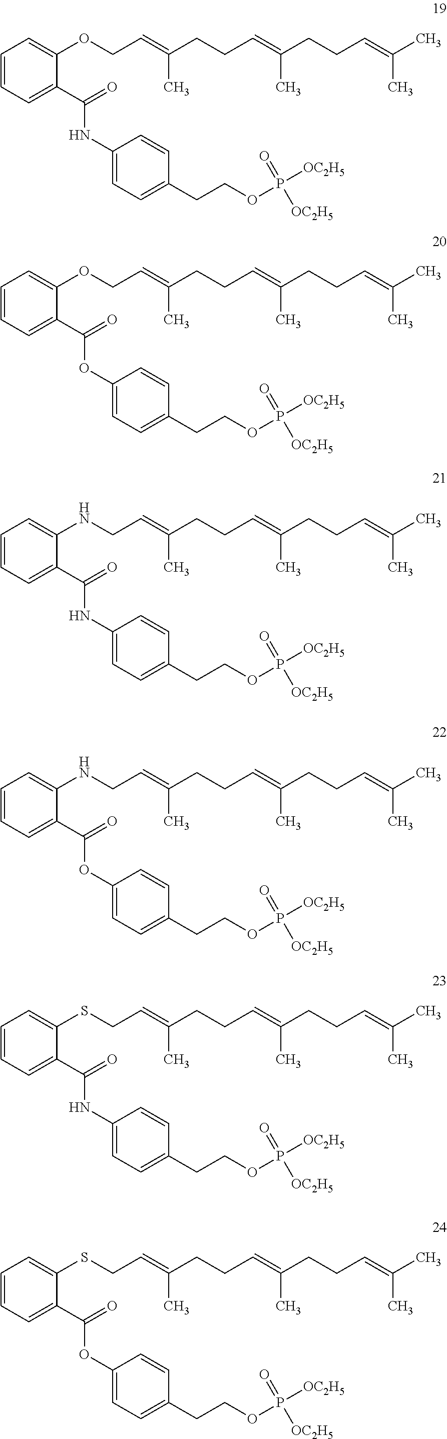 [SMILES: CCOP(=O)(OCC)OCCc1ccc(NC(=O)c2ccccc2OC/C=C(\C)CC/C=C(\C)CCC=C(C)C)cc1 ; CCOP(=O)(OCC)OCCc1ccc(OC(=O)c2ccccc2OC/C=C(\C)CC/C=C(\C)CCC=C(C)C)cc1 ; CCOP(=O)(OCC)OCCc1ccc(NC(=O)c2ccccc2NC/C=C(\C)CC/C=C(\C)CCC=C(C)C)cc1 ; CCOP(=O)(OCC)OCCc1ccc(OC(=O)c2ccccc2NC/C=C(\C)CC/C=C(\C)CCC=C(C)C)cc1 ; CCOP(=O)(OCC)OCCc1ccc(NC(=O)c2ccccc2SC/C=C(\C)CC/C=C(\C)CCC=C(C)C)cc1 ; CCOP(=O)(OCC)OCCc1ccc(OC(=O)c2ccccc2SC/C=C(\C)CC/C=C(\C)CCC=C(C)C)cc1]

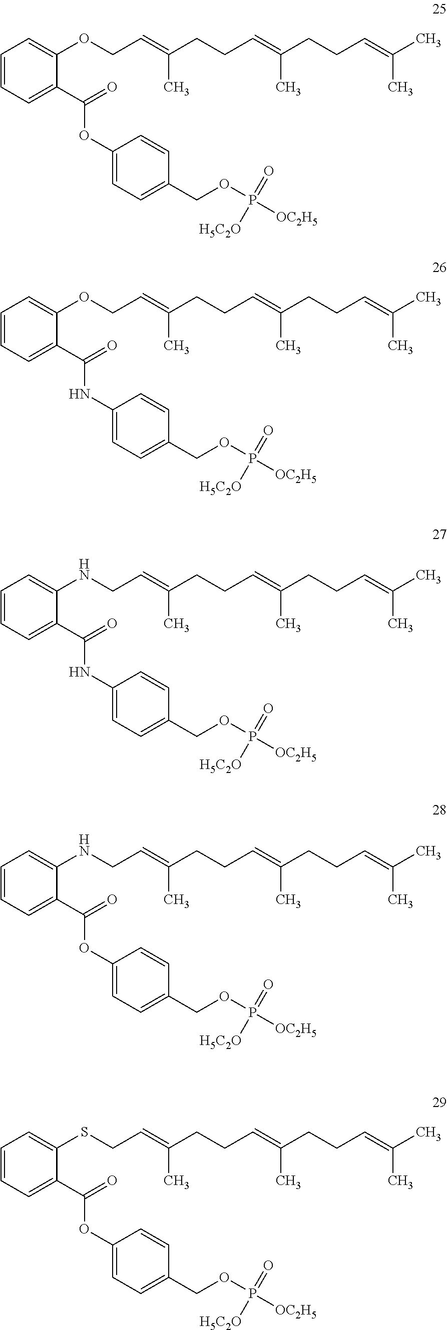

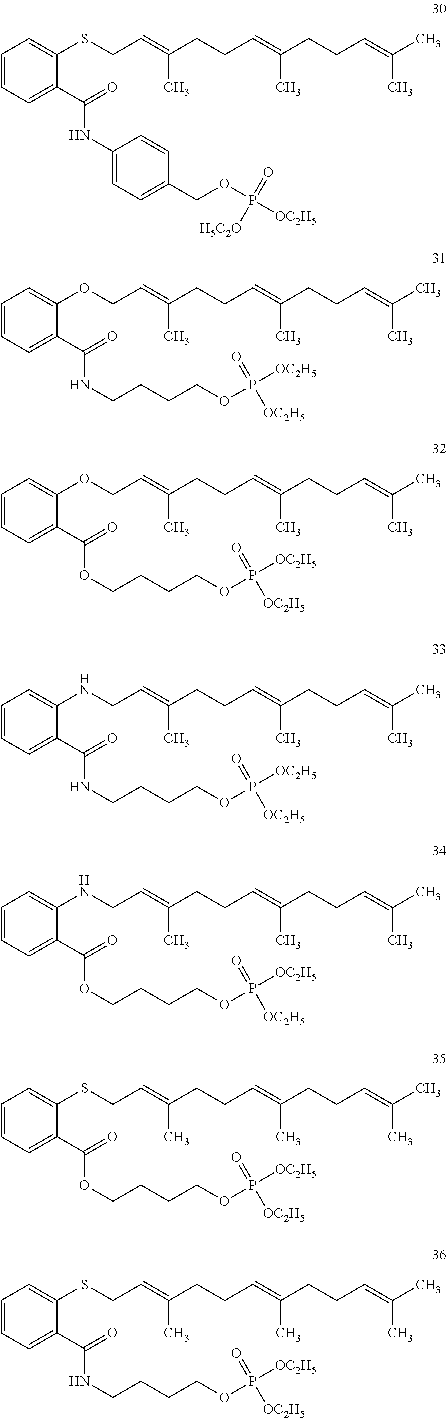

In a further embodiment of the invention, m is 0, the substituent X¹ is —O— and the substituents Z¹ and R⁹ are hydrogens. Thus, the corresponding compounds represented by Formula I are salicylic acid derivatives which include but are not limited to the following compounds 37 to 40.

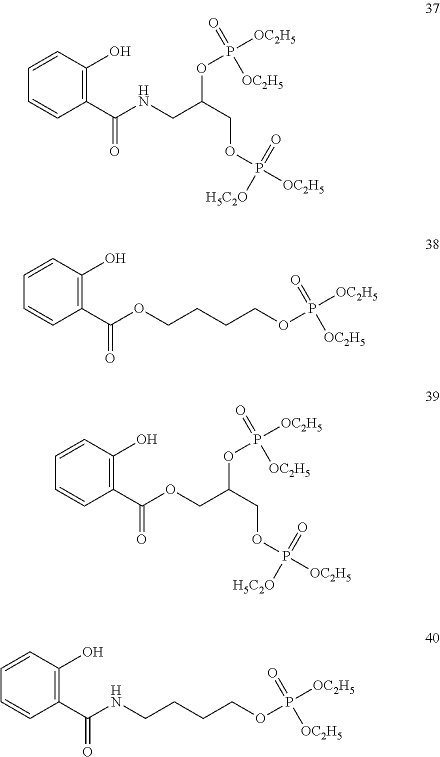

In still a further preferred embodiment, m is 1, the substituent X¹ is —O— and the substituents Z¹ and R⁹ are hydrogens. Thus, the compounds represented by Formula I are derivatives of dihydroxybenzoic acids including but not being limited to the following compounds 41 to 52.

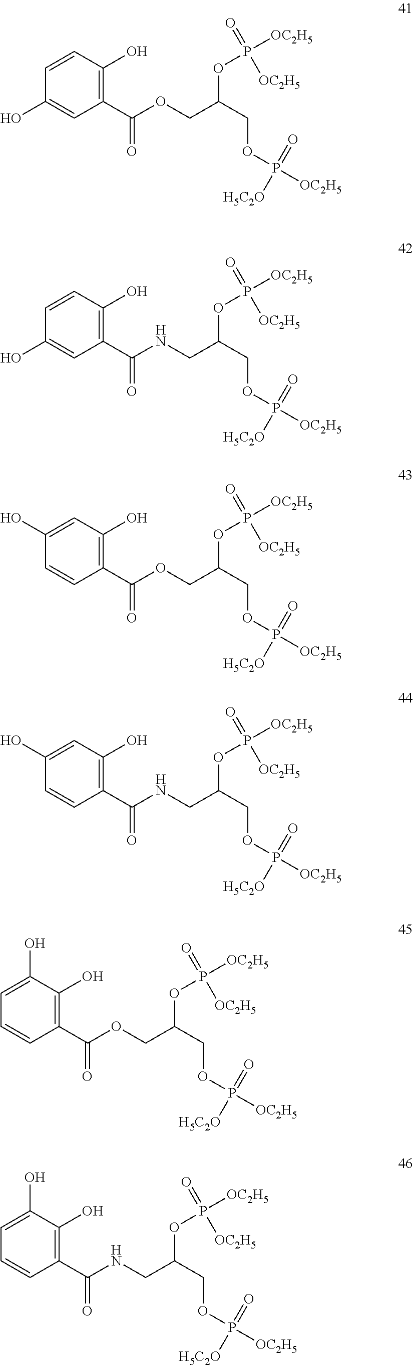

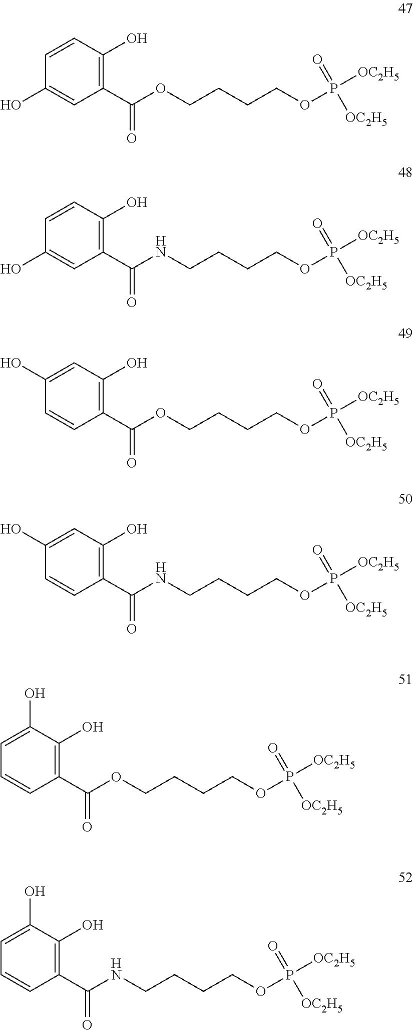 [SMILES: CCOP(=O)(OCC)OCCCCOC(=O)c1cc(O)ccc1O ; CCOP(=O)(OCC)OCCCCNC(=O)c1cc(O)ccc1O ; CCOP(=O)(OCC)OCCCCOC(=O)c1ccc(O)cc1O ; CCOP(=O)(OCC)OCCCCNC(=O)c1ccc(O)cc1O ; CCOP(=O)(OCC)OCCCCOC(=O)c1cccc(O)c1O ; CCOP(=O)(OCC)OCCCCNC(=O)c1cccc(O)c1O]

In still a further preferred embodiment of the present invention, the compound is represented by Formula II and the substituent R⁹ is hydrogen. Thus, the corresponding compounds are p-benzoquinone derivatives such as the compounds 53 to 56 shown below:

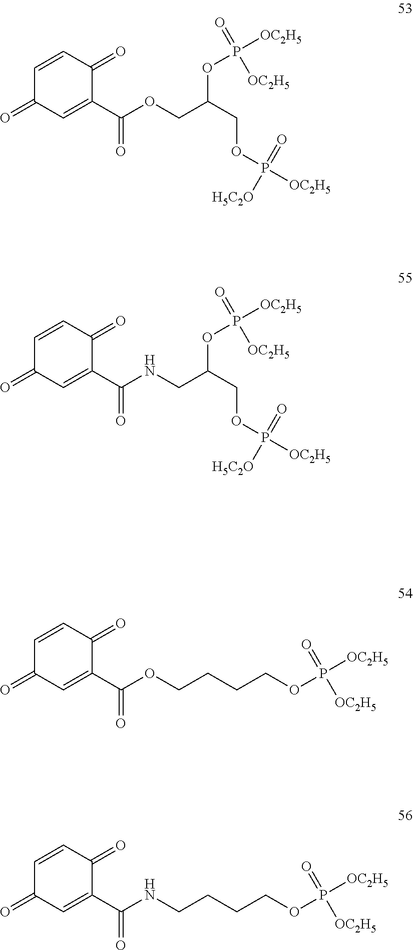

Some of the compounds of the present invention can comprise one or more stereogenic centers, and thus can exist in various isomeric forms, e.g. as stereoisomers and/or diastereomers. Thus, the compounds of Formulae I and II as well as pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. Moreover, when compounds of the invention exist in tautomeric forms, each tautomer is embraced herein.

In further embodiments, preferred compounds of the present invention may be described by the general Formula VIII:

A-D-Y Formula VIII

The compounds of Formula VIII include but are not limited to the following compounds:

| Compound | Compound | Compound | Compound | Compound | Compound |
|---|---|---|---|---|---|
| No. | structure | No. | structure | No. | structure |
| 38 | A¹-D¹-Y¹ | 57 | A¹-D¹-Y² | 58 | A¹-D¹-Y³ |
| 59 | A¹-D²-Y¹ | 60 | A¹-D²-Y² | 61 | A¹-D²-Y³ |
| 62 | A¹-D³-Y¹ | 63 | A¹-D³-Y² | 170 | A¹-D³-Y³ |
| 65 | A²-D¹-Y¹ | 66 | A²-D¹-Y² | 67 | A²-D¹-Y³ |
| 68 | A²-D²-Y¹ | 69 | A²-D²-Y² | 70 | A²-D²-Y³ |
| 71 | A²-D³-Y¹ | 72 | A²-D³-Y² | 73 | A²-D³-Y³ |
| 74 | A³-D¹-Y¹ | 75 | A³-D¹-Y² | 76 | A³-D¹-Y³ |
| 77 | A³-D²-Y¹ | 78 | A³-D²-Y² | 79 | A³-D²-Y³ |
| 80 | A³-D³-Y¹ | 81 | A³-D³-Y² | 82 | A³-D³-Y³ |
| 83 | A⁴-D¹-Y¹ | 84 | A⁴-D¹-Y² | 85 | A⁴-D¹-Y³ |
| 86 | A⁴-D²-Y¹ | 87 | A⁴-D²-Y² | 88 | A⁴-D²-Y³ |
| 89 | A⁴-D³-Y¹ | 90 | A⁴-D³-Y² | 91 | A⁴-D³-Y³ |
| 92 | A⁵-D¹-Y¹ | 93 | A⁵-D¹-Y² | 94 | A⁵-D¹-Y³ |
| 95 | A⁵-D²-Y¹ | 96 | A⁵-D²-Y² | 97 | A⁵-D²-Y³ |
| 98 | A⁵-D³-Y¹ | 99 | A⁵-D³-Y² | 100 | A⁵-D³-Y³ |
| 101 | A⁶-D¹-Y¹ | 102 | A⁶-D¹-Y² | 103 | A⁶-D¹-Y³ |
| 104 | A⁶-D²-Y¹ | 105 | A⁶-D²-Y² | 106 | A⁶-D²-Y³ |
| 107 | A⁶-D³-Y¹ | 108 | A⁶-D³-Y² | 109 | A⁶-D³-Y³ |
| 32 | A⁷-D¹-Y¹ | 110 | A⁷-D¹-Y² | 111 | A⁷-D¹-Y³ |
| 25 | A⁷-D²-Y¹ | 112 | A⁷-D²-Y² | 113 | A⁷-D²-Y³ |
| 20 | A⁷-D³-Y¹ | 114 | A⁷-D³-Y² | 115 | A⁷-D³-Y³ |
| 34 | A⁸-D¹-Y¹ | 116 | A⁸-D¹-Y² | 117 | A⁸-D¹-Y³ |
| 28 | A⁸-D²-Y¹ | 118 | A⁸-D²-Y² | 119 | A⁸-D²-Y³ |
| 22 | A⁸-D³-Y¹ | 120 | A⁸-D³-Y² | 121 | A⁸-D³-Y³ |
| 122 | A⁹-D¹-Y¹ | 123 | A⁹-D¹-Y² | 124 | A⁹-D¹-Y³ |
| 125 | A⁹-D²-Y¹ | 126 | A⁹-D²-Y² | 127 | A⁹-D²-Y³ |
| 128 | A⁹-D³-Y¹ | 129 | A⁹-D³-Y² | 130 | A⁹-D³-Y³ |
| 131 | A¹⁰-D¹-Y¹ | 132 | A¹⁰-D¹-Y² | 133 | A¹⁰-D¹-Y³ |
| 134 | A¹⁰-D²-Y¹ | 135 | A¹⁰-D²-Y² | 136 | A¹⁰-D²-Y³ |
| 137 | A¹⁰-D³-Y¹ | 138 | A¹⁰-D³-Y² | 139 | A¹⁰-D³-Y³ |
| 12 | A¹¹-D¹-Y¹ | 140 | A¹¹-D¹-Y² | 141 | A¹¹-D¹-Y³ |
| 10 | A¹¹-D²-Y¹ | 142 | A¹¹-D²-Y² | 143 | A¹¹-D²-Y³ |
| 8 | A¹¹-D³-Y¹ | 144 | A¹¹-D³-Y² | 145 | A¹¹-D³-Y³ |
| 6 | A¹²-D¹-Y¹ | 146 | A¹²-D¹-Y² | 147 | A¹²-D¹-Y³ |
| 4 | A¹²-D²-Y¹ | 148 | A¹²-D²-Y² | 149 | A¹²-D²-Y³ |
| 2 | A¹²-D³-Y¹ | 150 | A¹²-D³-Y² | 151 | A¹²-D³-Y³ |
| 152 | A¹³-D¹-Y¹ | 153 | A¹³-D¹-Y² | 154 | A¹³-D¹-Y³ |
| 155 | A¹³-D²-Y¹ | 156 | A¹³-D²-Y² | 157 | A¹³-D²-Y³ |
| 158 | A¹³₋D³-Y¹ | 159 | A¹³-D³-Y² | 160 | A¹³-D³-Y³ |
| 161 | A¹⁴-D¹-Y¹ | 162 | A¹⁴-D¹-Y² | 163 | A¹⁴-D¹-Y³ |
| 164 | A¹⁴-D²-Y¹ | 165 | A¹⁴-D²-Y² | 166 | A¹⁴-D²-Y³ |
| 167 | A¹⁴-D³-Y¹ | 168 | A¹⁴-D³-Y² | 169 | A¹⁴-D³-Y³ |

whereby

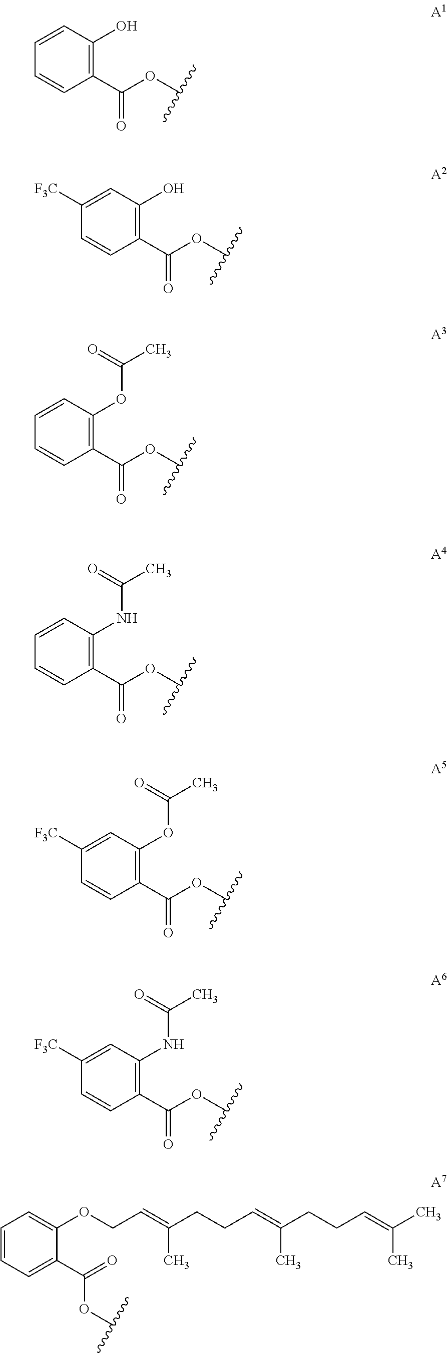

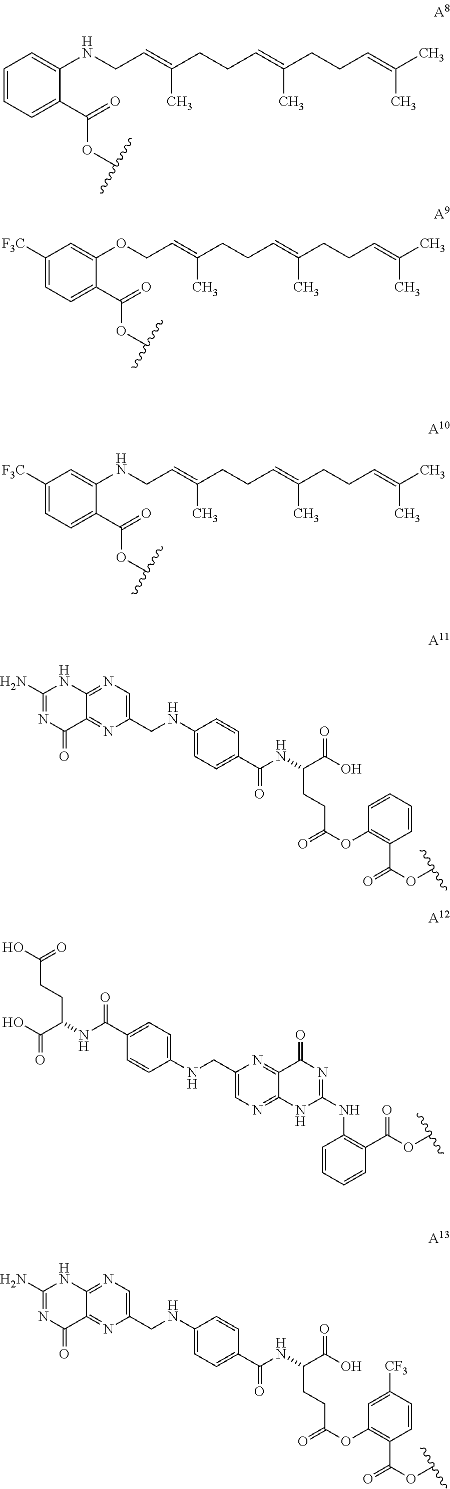

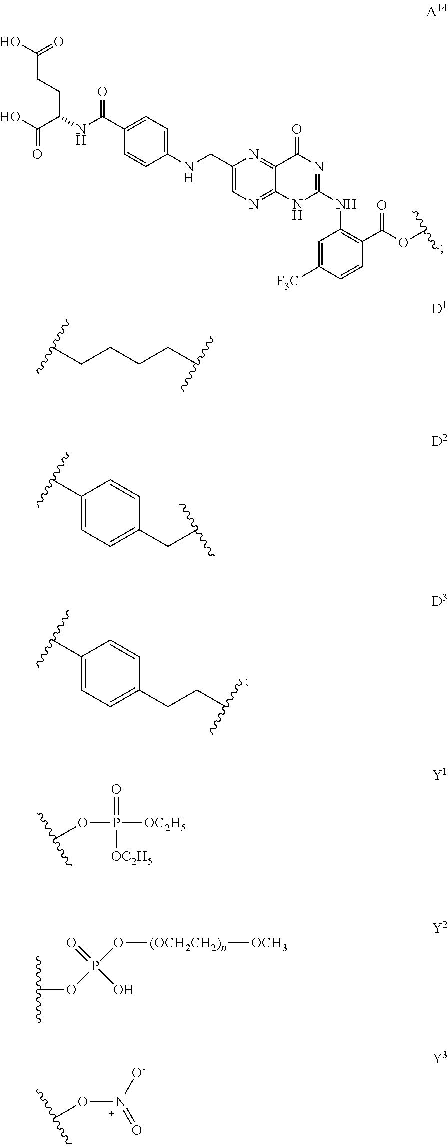

and n is preferably between 40 and 50.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g. racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

The phrase, “pharmaceutically acceptable derivative”, as used herein, denotes any pharmaceutically acceptable salt, ester, or salt or cocrystal of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others prodrugs. A prodrug is a derivative of a compound, usually with significantly reduced pharmacological activity, which contains at least one additional moiety, which is susceptible to removal in vivo yielding the parent molecule as the pharmacologically active species. An example of a prodrug is an ester, which is cleaved in vivo to yield a compound of interest. Prodrugs of a variety of compounds, and materials and methods for derivatizing the parent compounds to create the prodrugs, are known and may be adapted to the present invention. Certain exemplary pharmaceutical compositions and pharmaceutically acceptable derivatives will be discussed in more detail herein below.

According to the present invention, the compounds of Formulae I and II possess anti-cancer activity, anti-inflammatory activity, analgesic activity and/or antipyretic activity.

In one embodiment of the present invention, the compounds represented by Formulae I and II and pharmaceutical compositions thereof have anti-cancer activity. Thus, the compounds represented by Formulae I and II and pharmaceutical compositions thereof inhibit the growth of human or animal cancer cell lines such as HT-29 in in vitro tests and have IC₅₀ value of preferably less than 300 μM, more preferred of less than 100 μM, particularly preferred of less than 70 μM. The tests are preferably carried out as specified in S. Joseph et al. (Molecular Medicine Reports 2011, 4, 891-899).

In some embodiments, the compounds represented by Formulae I and II and pharmaceutical compositions thereof possess anti-inflammatory activity. Thus, the compounds represented by Formulae I and II and pharmaceutical compositions thereof can reduce the levels of inflammatory cytokines such as tumor necrosis factor-α (TNF-α) by at least 50% in in vivo tests with female LEW/CrlBR Lewis rats when given in a daily dosage of preferably no more than 500 mg/kg, more preferred of no more than 300 mg/kg, particularly preferred of no more than 100 mg/kg. The tests are preferably performed according to the procedure by L. Huang et al. (Br. J. Pharmacol. 2011, 162, 1521-1533).

The compounds of the present invention have a high in vivo stability. Preferably, the concentration the compounds of Formula I in blood plasma of an animal after 3 h is at least 30% of its initial concentration, more preferred at least 40% of its initial concentration, particularly preferred at least 50% of its initial concentration. The corresponding tests can be carried out with animals such as mice according to the method described by Xie et al. (Br. J. Pharmacol. 2012, 165, 2152-2166).

In addition, the compounds of the present invention have cellular uptake values, which can be determined by using cancer cells, for instance human non-small cell lung cancer cells A549 and subsequently assaying their intracellular levels by high performance liquid chromatography (HPLC). Preferably, the cellular uptake values of the compounds are higher than 0.1 nmol/mg protein, more preferred higher than 1.0 nmol/mg protein, even more preferred higher than 10.0 nmol/mg protein and particularly preferred higher than 50.0 nmol/mg protein.

Preferably, the compounds of Formulae I and II have n-octanol-water partition coefficient (log P) value higher than 1, more preferred higher than 2 and particularly preferred higher than 3. Log P is defined as ratio of concentrations (mol/volume) of the compounds of Formula I in n-octanol and in water. Suitable methods for the measurement of n-octanol-water coefficients are, for instance described in Octanol-Water Partition Coefficients: Fundamentals and Physical Chemistry, John Wiley and Sons Ltd., 1997, ISBN: 0-417-97397 1. Both solvents are mutually saturated before the measurement. At equilibrium the n-octanol phase contains 2.3 mol/l of water and the aqueous phase contains 4.5×10⁻³ mol/l of n-octanol. The measurement is carried out at the isoelectric point of the compound of Formula I at temperature of 25° C. The log P of the compounds of Formula I is preferably determined by the shake-flask method, which is, for example, described in the review of J. Sangster (J. Phys. Chem. Ref. Data 18, 1989; 3, 1111-1227). The measurement is carried out under the conditions described by T. Fujita et al. (J. Am. Chem. Soc. 1964, 86, 5175-5180) and the concentration of the compounds in each of the two phases is determined by HPLC.

The pharmaceutical composition containing the compound of Formulae I or II is useful in the treatment and/or prevention of cancer. “Cancer” as used herein refers to an uncontrolled growth of cells which interferes with the normal functioning of the bodily organs and systems. Cancers include, but are not limited to basal cell carcinoma, biliary tract cancer; bladder cancer; bone cancer; brain and other central nervous system (CNS) cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemias, including hairy cell leukemia; liver cancer; lung cancer (e.g. small cell and non-small cell); lymphomas including Hodgkin's and non-Hodgkin's lymphomas; melanoma; myeloma; neuroblastoma; oral cavity cancer (e.g. lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

In yet another embodiment, the pharmaceutical composition containing the compound of Formulae I and II is useful in the treatment and/or prevention of cancer and precancerous conditions, including but not limited to benign prostatic hypertrophy, colon adenomas, actinic keratosis and various premalignant conditions of the lung, breast and pancreas as well as metastases thereof.

A metastasis is a region of cancer cells, distinct from the primary tumor location resulting from the dissemination of cancer cells from the primary tumor to other parts of the body. In one embodiment, the cancer is melanoma (primary or metastatic). In one embodiment, the cancer is breast cancer. In another embodiment, the cancer is lung cancer. In a further embodiment, the cancer is pancreas cancer. In a further embodiment, the cancer is colon cancer. In a further embodiment, the cancer is skin cancer.

The compounds of the invention are useful in the treatment of the above-mentioned cancers. They are particularly suited for treating neoplastic and pre-neoplastic diseases of human and animal including but not limited to, for example, benign prostatic hypertrophy, prostate cancer, colon adenomas and colon cancer, cancer or preneoplastic conditions of the lung, breast, brain, pancreas and liver, lymphomas and leukemias.

In a particularly preferred embodiment, the compounds of Formulae I and II are used for the treatment of a cancer with a mutant RAS gene, for instance of a pancreatic cancer. The term “mutant RAS gene” as used herein relates to a H-ras, K-ras or N-ras genes bearing at least one mutation, such as a single base mutation. Said genes encode mutant proteins which are distinct from the corresponding wild-type proteins. Mutant RAS genes are detectable by methods known in the prior art, for instance as described in U.S. Pat. No. 6,090,546.

In a further aspect, the invention is directed to a pharmaceutical composition comprising a compound of Formulae I or II, as described generally herein, and a pharmaceutically acceptable excipient. In a specific embodiment, the composition is useful in the treatment of human and animal inflammation and inflammation related diseases including but not limited to rheumatologic diseases such as rheumatoid arthritis, osteoarthritis and Sjogren's syndrome; cardiovascular diseases, such as coronary artery disease, peripheral vascular disease and hypertension; neurodegenerative diseases such as Alzheimer's disease and its variants or cerebrovascular diseases; and autoimmune diseases such as lupus erythematosus; and other conditions characterized by chronic inflammation of organs such as the lung, such as chronic bronchitis or the sinuses, such as chronic sinusitis and inflammatory conditions of the gut such as inflammatory bowel disease; cardiovascular diseases, for example, coronary artery disease, peripheral vascular disease and hypertension; neurodegenerative diseases, for example, Alzheimer's disease and its variants or cerebrovascular diseases; and autoimmune diseases such as lupus erythematosus; other conditions characterized by chronic inflammation of organs such as the lung, such as chronic bronchitis or the sinuses, such as chronic sinusitis and also for acute inflammation, such as but not limited to dental/periodontal, skin, intestinal, ear, nose and throat acute inflammatory conditions, acute bronchitis, acute enteritis, hepatitis and pancreatitis.

The compounds and pharmaceutical compositions of the present invention are further useful for alleviating (or mitigating) or treating a pain, for example, a chronic pain (particularly, a neuropathic pain) effectively. The compounds and pharmaceutical compositions of the present invention can, for instance, be administered as an injectable therapy adapted for one or more applications selected from a group consisting of subcutaneous, caudal, epidural, intramuscular, intradural, intraspinous and peripheral nerve blockade. They can further be formulated by entrapping in liposomes, lipid and polymeric micelles, dendrimers, solid lipid nanoparticles or other nanoparticles. The compounds and pharmaceutical compositions of the present invention are preferably capable of providing analgesic effect for at least 2 hours, more preferred for at least 4 hours, yet even more preferred for at least 6 hours.

Another aspect of the present invention provides novel compounds of Formulae I and II and pharmaceutical compositions thereof for preventing and/or treating hyperthermia, fever, or pyrexia in mammalian subjects. In various embodiments, the compounds and compositions of the present invention are effective for preventing elevation of body temperature above a normal body temperature range, and/or for lowering body temperature that has elevated above normal body temperature range in mammalian subjects suffering from impairment of thermal homeostasis.

By administering the antipyretic compound of the invention in a suitable prophylactic or therapeutic treatment protocol, subjects presenting with, or at elevated risk for, neuroleptic malignant syndrome or malignant hyperthermia can be effectively treated. Treatment of these conditions using the compounds and pharmaceutical compositions provided herein will reduce or prevent elevated temperatures in these subjects, and will often additionally substantially prevent or alleviate one or more of the above-identified symptoms associated with the subject condition as well.

Antipyretic agents are provided for effective management, prophylaxis, and/or treatment of various forms of “hot flashes” that occur in mammalian subjects. Hot flashes are most commonly associated with menopause, however, they may also be drug induced (for example by anti-estrogen compounds such as tamoxifen, toremifen and raloxifen), or triggered by removal of estrogen-producing tissues (e.g. after abdominal hysterectomy and bilateral salpingo-oopherectomy. As used herein, the term “hot flash” refers to any sudden, typically brief, sensation of heat, which often appears to affect the entire body, and may further be accompanied by secondary symptoms, including sweating, palpitations, and/or red blotching of the skin. In the exemplary case of menopausal hot flashes (i.e. menopausal, post-menopausal, and perimenopausal hot flashes) the antipyretic agents of Formulae I and II provided by the present invention are effective to substantially prevent or alleviate one or more of the foregoing symptoms.

Antipyretic effectiveness of the compounds of the present invention in this context may be demonstrated, for example, by a reduction in the number of hot flashes experienced by test versus control subjects, wherein the number of hot flashes of treated menopausal subjects may be reduced, for example, to fewer than 5 per day, fewer than 3 per day, fewer than 2 per day, fewer than 1 per day, or eliminated altogether. Alternatively, effectiveness may be demonstrated by a number of other numerical evaluation and scale rating systems including, but not limited to, the Kupperman Menopausal Index, the Menopause Rating Scale, Montgomery-Asberg Depression Rating Scale, the Hamilton Anxiety Rating Scale and the Hamilton Depression Rating Scale. Using the Hamilton Depression Rating Scale, for example, a score of 10-13 indicates mild depression; 14-17 mild to moderate depression; >17 moderate to severe depression. In the Hamilton Anxiety Rating Scale, mild anxiety is 18-24, moderate anxiety is 25-29 and severe anxiety would be any number over 30. With the Kupperman Menopausal Index is an assessment system that involves grading major menopausal symptoms from 0 (not present) to 3 (severe) and using the total score to quantify severity symptoms. The symptoms include hot flashes, depression, headache, palpitations, joint pain, loss of concentration, sleep disturbance, profuse perspiration, nervousness and irritability.

Hyperthermia is also common in cancer patients, either through infection, tumor development (causing paraneoplastic fever), drugs (allergic or hypersensitivity reactions), blood product transfusion, and graft-versus-host disease (GVHD). Paraneoplastic fever, or fever caused by tumors, is particularly common in patients presenting with lymphoma and renal cell carcinoma. These and other subjects are effectively treated, prophylactically and/or therapeutically, by administering to the subject an antipyretic effective amount of an antipyretic agent of Formula I or II sufficient to prevent or reduce temperature elevation, as noted above, or to prevent or alleviate one or more related hyperthermic response(s) and/or one or more symptom(s) secondary or attendant to hyperthermia in the subject.

In a specific embodiment, the invention is directed to a method for obtaining a pharmaceutical composition, comprising formulating the compounds of the present invention into a composition comprising the compound of the present invention and one or more pharmaceutically acceptable carrier or excipient. The invention is further directed to uses of the compounds of the present invention for manufacturing a medicament.

### Compositions

As discussed above, this invention provides novel compounds that have biological properties and pharmacological activity useful for the treatment of any of a number of conditions or diseases generally characterized by abnormal inflammation, or prophylaxis in instances wherein a risk of appearance of such conditions or diseases is present as well as for the treatment and/or prevention of cancer. Moreover, certain compounds known in the art have been newly identified as having activity likewise useful in the prophylaxis or treatment of abnormal inflammation and cancers, and the invention is also directed to anti-inflammation and anti-cancer compositions comprising such compounds.

Accordingly, in another aspect of the present invention, pharmaceutical compositions are provided, which comprise any one of the compounds described herein (or a pharmaceutically acceptable salt, cocrystal or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional co-administered therapeutic agents or included in a pharmaceutical composition with a compound of this invention may be an approved anti-inflammation agent, or it may be any one of a number of agents undergoing approval in the Food and Drug Administration that ultimately obtain approval for the treatment of any disorder related to inflammation. Such additional therapeutic agents may also be provided to promote the targeting of the compounds of the invention to the desired site of treatment, or may increase their stability, increase their plasma half-life, and further improve their biodistribution and pharmacokinetics. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts or cocrystals of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt or cocrystals” refers to those salts or cocrystals which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts or cocrystals of amines, carboxylic acids, and other types of compounds, are well known in the art. For example, S. M. Berge, et al. describe pharmaceutically acceptable salts in detail in J. Pharmaceutical Sciences 1977, 66, 1-19, incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts and coformer molecules for cocrystal formation, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are transformed in vivo to yield the parent compound of the above formula, for example by hydrolysis in blood. A thorough discussion is provided in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal® 53 MCT, Phosal® 50 PG, Phosal® 75 SA, Phospholipon® 90H, Phospholipon® 90G and Phospholipon® 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to pharmaceutically acceptable, emulsion preconcentrates the so-called self-emulsifying drug delivery systems (SEDDS), emulsions, microemulsions, nanoemulsions, solutions, suspensions, syrups and elixirs. In addition to the active compounds, the liquid dosage forms may contain inert diluents commonly used in the art such as, for example, water or other solvents, solubilizing agents, oils and emulsifiers. Suitable solvents include ethyl alcohol, isopropyl alcohol, ethyl carbonate, ethyl acetate, benzyl alcohol, benzyl benzoate, propylene glycol, 1,3-butylene glycol, dimethylformamide, glycerol, tetrahydrofurfuryl alcohol and polyethylene glycols. Oil components include soybean, cottonseed, groundnut (peanut), corn, germ, olive, castor, almond, sesame and fish oil, and mixtures thereof. Surfactants suitable for the compositions of the present invention, include mono- and/or diglycerides of fatty acids and their acetic, succinic, lactic, citric and/or tartaric esters, propylene glycol fatty acid esters, mixtures of propylene glycol esters and glycerol esters, polyethoxylated fatty acids, PEG-fatty acid diesters, PEG-fatty acid mono- and di-ester mixtures, polyethylene glycol and glycerol fatty acid esters, alcohol-oil trans-esterification products, polyglycerized fatty acids, polyethylene glycol sorbitan fatty acid esters, polyethylene glycol alkyl ethers, sorbitan fatty acid esters, lower alcohol fatty acid esters, sugar esters, vitamin E esters, such as α-tocopherol-polyethylene-glycol-1000-succinate, poloxyethylene-polyoxypropylene block copolymers, known as Pluronics® also known as Poloxamers, lecithin, C₆₋₂₂ fatty acids and salts and mixtures thereof. Besides inert diluents, the oral compositions can also include adjuvants such as wetting agents, emulsifying and suspending agents, sweetening, flavoring, and perfuming agents.

The oral liquid compositions of the present invention can be filled into hard or soft gelatin capsule or as bulk oral solutions in a bottle. These dosage forms can be manufactured by well-established methods that are known in the art. The liquid-filled capsules can be further coated with enteric polymers for releasing the active in the small intestine or colon.

Injectable preparations, for example, sterile injectable aqueous or oleaginous suspensions and dispersions may be formulated according to the known art using suitable dispersing or wetting agents and suspending agents. The sterile injectable preparation may be a solution, suspension or emulsion in a nontoxic parenterally acceptable diluent or solvent. Among the acceptable aqueous vehicles and solvents that may be employed are water, Ringer's solution, U.S.P. and isotonic sodium chloride solution. In addition, vegetable oils phospholipids and surfactants form the list provided above and approved for parenteral drug administration are conventionally employed as a solvents, suspending or dispersing agents.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of Formulae I or II, it is often desirable to slow the absorption of these compounds from subcutaneous or intramuscular injection.

This may be accomplished by the use of a liquid suspension or crystalline or amorphous material with poor water solubility. The rate of absorption of the compounds of Formulae I or II then depends upon its rate of dissolution that, in turn, may depend upon crystal size and crystalline form. Alternatively, delayed absorption of a parenterally administered compounds of Formulae I or II is accomplished by dissolving or suspending the compounds in an oil vehicle. Injectable depot forms are made by forming microencapsulated matrices of the compounds in biodegradable polymers such as polylactide-polyglycolide. Depending upon the ratio of drug to polymer and the nature of the particular polymer employed, the rate of compound release can be controlled. Examples of other biodegradable polymers include poly(orthoesters) and poly(anhydrides). Depot injectable formulations are also prepared by entrapping the compounds in liposomes or microemulsions, preferably into liposomes, which are more biocompatible.

In particular, the compounds represented by Formulae I and II above are highly suitable for incorporation into liposomes. The resulting compositions are particularly useful for the treatment and/or prevention of cancers such as lung cancer and colon cancer. Preferred liposome compositions are those which in addition to other phospholipids, incorporate pegylated phospholipids, such as DSPE-PEG2000, and exhibit long circulation times by avoiding uptake and clearance by the reticuloendothelial system (RES) and thus, are able to reach and treat solid tumors in the body.

Compositions to deliver the compounds of Formulae I or II directly to the colon—for example, tablets or capsules incorporating enteric coating pH-sensitive polymers, such as those available by the trade name Eudragit® and/or polysaccharides, such as pectin, from which the active agent is released into the colon by a pH-dependent mechanism and/or through degradation by the bacteria present in the colon or by other mechanism, ensuring exclusive or predominant colonic delivery of said compounds. Other means for colonic delivery include suppositories and enemas.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include but are not limited to capsules, tablets, pills, pellets, powders, and granules. In such solid dosage forms, the compound of Formula I or II is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, pills and pellets, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, pellets, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The compounds of Formulae I or II can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the compounds of Formulae I or II may be admixed with at least one inert diluent such as sucrose, lactose and starch. Such dosage forms may also comprise, as in normal practice, additional substances other than inert diluents, e.g. tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include but are not limited to polymeric substances and waxes.

The present invention encompasses pharmaceutically acceptable topical formulations of inventive compounds of Formulae I and II. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to solvents (e.g. alcohols, polyhydric alcohols, water), creams, lotions, ointments, oils, plasters, liposomes, powders, emulsions, microemulsions, and buffered solutions (e.g. hypotonic or buffered saline) or any other carrier known in the art for topically administering pharmaceuticals. A more complete listing of art-known carriers is provided by reference texts that are standard in the art, for example, Remington's Pharmaceutical Sciences, 16^{th} Edition, 1980 and 17^{th} Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to alcohols, quaternary amines, organic acids, parabens, and phenols. Suitable antioxidants include, but are not limited to ascorbic acid and its esters, sodium bisulfite, butylated hydroxytoluene, butylated hydroxyanisole, tocopherols, and chelating agents like EDTA and citric acid. Suitable moisturizers include, but are not limited to glycerin, sorbitol, polyethylene glycols, urea, and propylene glycol. Suitable buffering agents for use with the invention include, but are not limited to citric, hydrochloric, and lactic acid buffers. Suitable solubilizing agents include, but are not limited to quaternary ammonium chlorides, cyclodextrins, benzyl benzoate, lecithin, poloxamers and polysorbates. Suitable skin protectants that can be used in the topical formulations of the invention include, but are not limited to vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in Formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. P., Yum S. I. (Eds.), Interpharm Press Inc., Buffalo Grove, III. (1997). In certain exemplary embodiments, penetration agents for use with the invention include, but are not limited to triglycerides (e.g. soybean oil), aloe compositions (e.g. aloe-vera gel), ethyl alcohol, isopropyl alcohol, octolyphenylpolyethylene glycol, oleic acid, polyethylene glycol 400, propylene glycol, n-decylmethylsulfoxide, fatty acid esters (e.g. isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. Gel compositions for applying the active compounds of the present invention to the skin, particularly those incorporating Pluronic® surfactants also known as poloxamers, such as Pluronic P123, are preferred. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound of Formula I or II in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammation or anti-cancer agent), or they may achieve different effects (e.g. control of any adverse effects).

In certain embodiments, the pharmaceutical compositions of the present invention further comprise one or more additional therapeutically active ingredients (e.g. anti-cancer, anti-inflammatory, antipyretic and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs.

In certain embodiments, the compounds of the present invention can be covalently or non-covalently bound to, for example, polyethylene glycol or other similar molecules to make them suitable for administration to the patient either in one of the forms described above or using nanodevices. In a preferred embodiment of the present invention, the compounds can be formulated into nanoparticles to optimize their delivery, intracellular targeting and therapeutic effect. Particularly preferred nanoparticulate compositions of the compounds are liposomes, solid lipid nanoparticles and polymeric micelles, particularly PEO-b-PLA [poly(ethylene oxide)-b-poly(lactid acid) micelles and dendrimers.

### Uses and Methods of Treatment

As discussed above, certain of the compounds represented by Formulae I and II exhibit activity generally as inhibitors of pain, inflammation and/or cancer. Thus, in certain embodiments, compounds of the invention are useful for the treatment of any of a number of conditions or diseases in which inflammation, in particular chronic inflammation is the cause of or relates to the onset or continued occurrence of the disease or condition, such as but not limited to rheumatologic diseases such as rheumatoid arthritis and Sjogren's syndrome; cardiovascular diseases, for example, coronary artery disease, peripheral vascular disease and hypertension; neurodegenerative diseases, for example, Alzheimer's disease and its variants or cerebrovascular diseases; and autoimmune diseases such as lupus erythematosus; other conditions characterized by chronic inflammation of organs such as the lung, such as chronic bronchitis or the sinuses, such as chronic sinusitis. Moreover, compounds of the present invention are also useful for the treatment of cancers, in particular colon cancer, pancreas cancer, brain cancer and lung cancer.

Accordingly, in another aspect of the invention, methods for the treatment of inflammation-related disorders and/or cancer are provided comprising administering a therapeutically effective amount of a compound of Formula I or II to a subject in need thereof. In certain embodiments, a method for the treatment of related disorders is provided comprising administering a therapeutically effective amount of an inventive compound, or a pharmaceutical composition comprising an inventive compound to a subject in need thereof, in such amounts and for such time as is necessary to achieve the desired result.

The invention is also directed to the use of any compound of Formula I or II for the preparation of a medicament for administration to a human or animal patient in need thereof, to inhibit or block inflammation and/or inhibit the growth of cancer. Such compounds preferably are administered once a precancerous condition, an inflammation-related disease or an inflammatory condition that may predispose to disease or cancer has been diagnosed in the patient, optionally in combination with other anti-inflammation agents or other anti-cancer agents such as those that maintain therapeutic levels of the compounds within the body. Compounds of the invention also may be administered after other therapies have been tried and failed, and may be administered prophylactically.

In certain embodiments, the uses and methods of the invention involve the administration of a therapeutically effective amount of the compound or a pharmaceutically acceptable derivative thereof to a subject (including, but not limited to a human or animal, including livestock, domesticated or zoo animals) in need thereof.

It will be appreciated that the compounds and compositions, according to the method of the present invention, may be administered using any amount and any route of administration effective for the treatment of conditions or diseases in which anti-inflammation, anti-cancer, analgesic, antipyretic or related activities have a therapeutically useful role. Thus, the expression “effective amount” as used herein, refers to a sufficient amount of agent to inhibit inflammation or to exhibit the desired therapeutic effect. The exact amount required will vary from subject to subject, depending on the species, age, and general condition of the subject, the severity of the infection, the particular therapeutic agent, its mode of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with appropriate pharmaceutically acceptable carriers in a desired dosage form, the pharmaceutical compositions of this invention can be administered to a human or animal subject orally, rectally, parenterally (intravascularly, intramuscularly, intraperitoneally, subcutaneously), intracisternally, intravaginally, topically in the form of a gel, cream, ointment, lotion or drops, bucally in the form of a gel or tablet, or the like, depending on the location and extent of the disease being treated. In certain embodiments, the compounds of the invention may be parenterally administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. In other embodiments, compounds of the invention may be administered orally or rectally at dosage levels of about 0.01 mg/kg to about 100 mg/kg, from about 0.05 mg/kg to about 50 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, for instance if the patient suffers from Alzheimer's disease, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXAMPLES

All experiments were performed under atmospheric pressure of 100.3±5 kPa and room temperature unless stated otherwise. The term “room temperature” refers to a temperature of 20±2° C.

### Example 1

### Anti-Cancer Activity and Pharmacokinetics of Phospho-Aspirin (PA)

The ability of phospho-aspirin (PA) to prevent breast cancer (BC) was evaluated using MDA-MB-231 human BC cells xenografted into one of the mammary glands of nude mice (orthotopic xenografts). PA was administered orally, 120 mg/kg/d 1 wk prior to inoculating the cells (the standard prevention protocol); acetylsalicylic acid (ASA) was given at an equimolar dose of 40 mg/kg/d. This was the highest dose of ASA that these mice could tolerate on a long-term basis. The dose of PA represents <10% of its maximum tolerated dose (MTD≧1,600 mg/kg), but was chosen so that a comparison between PA and ASA was possible.

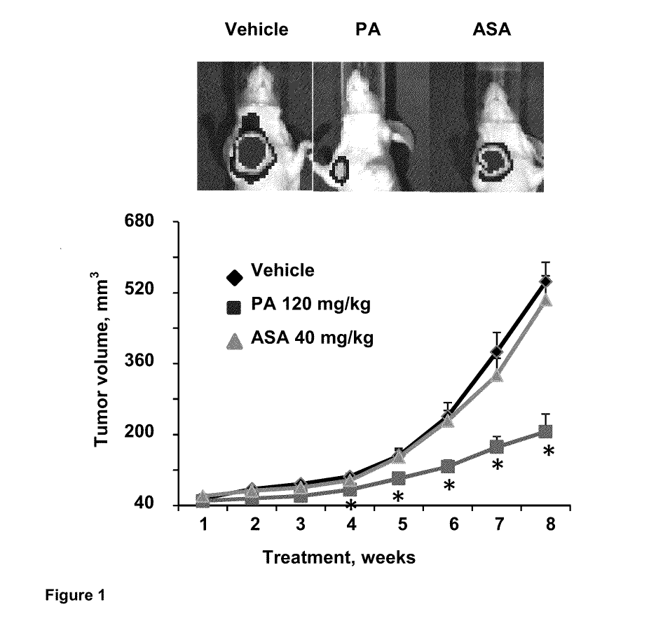

FIG. 1 illustrates the growth of orthotopic MDA-MB231 xenografts treated with PA or ASA, starting 1 wk prior to cell implantation. Cells were stably transferred with luciferase allowing imaging of the xenografts (upper images). Lower diagram: tumor volume, mm³. Volume calculations were based on luminescence and caliper measurements that agreed closely. *, p<0.001-05.

PA displayed a strong chemoprevention effect (FIG. 1). Of the 20 treated mice: 20% had no tumors; 30% had tumors <100 mm³, while the smallest tumor in controls was 326 mm³ and the average tumor volume was reduced by 62% compared to controls. In sharp contrast, ASA showed no effect on BC xenograft growth.

The levels of PA and some of its metabolites in blood and xenografts from these mice were determined by HPLC according to the methods described by G. Xie et al. (Br J Pharmacol. 2012; 167(1):222-32).

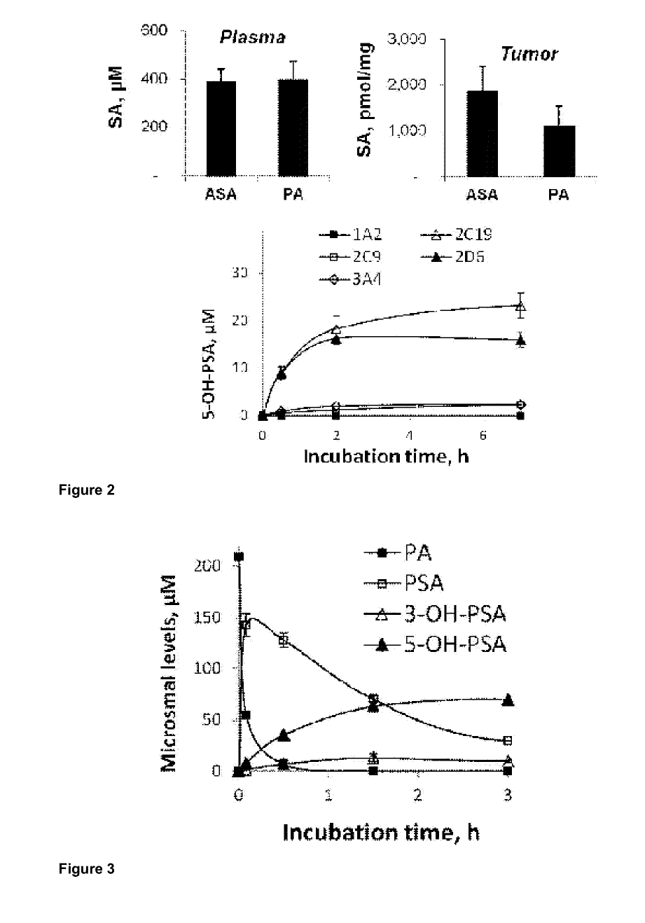

FIG. 2 shows the measured levels of PA metabolites in plasma and tumors; and the effect of CYP isoforms. Upper panels: Only salicylic acid (SA) was detected in the plasma and xenograft tumors of mice treated with PA or ASA (study shown in FIG. 1). Differences in SA levels were not significant. Lower panel: In vitro metabolism of PSA by human CYPs. PSA was incubated with each of the indicated CYP isoforms and its rate of conversion to 5-OH-PSA was monitored. Only 2C19 and 2D6 showed appreciable activity.

No intact PA in either plasma or xenografts was detected; salicylic acid (SA) being the only measurable metabolite (FIG. 2). Moreover, there was no difference in SA levels between the two groups. These results were an unexpected finding based on what is conventionally understood regarding drug action. Thus we entertained the possibility that additional agents may be involved in the differential action of these two compounds and attempted to identify them; in this effort we took into consideration the findings of our comprehensive work on the metabolism of PA, described in Example 2.

### Example 2

### Formation of Phospho-Aspirin (PA) Metabolites

Using standard approaches, included human and animal liver microsomes, cultured breast cancer cells and mice, as well as established analytical methods, the metabolism of PA was investigated. Results are summarized in Scheme 3 (Xie G, Wong C C, Cheng K W, Huang L, Constantinides P P, Rigas B. In Vitro and In Vivo Metabolic Studies of Phospho-aspirin (MDC-22). Pharm Res. 2012, in press).

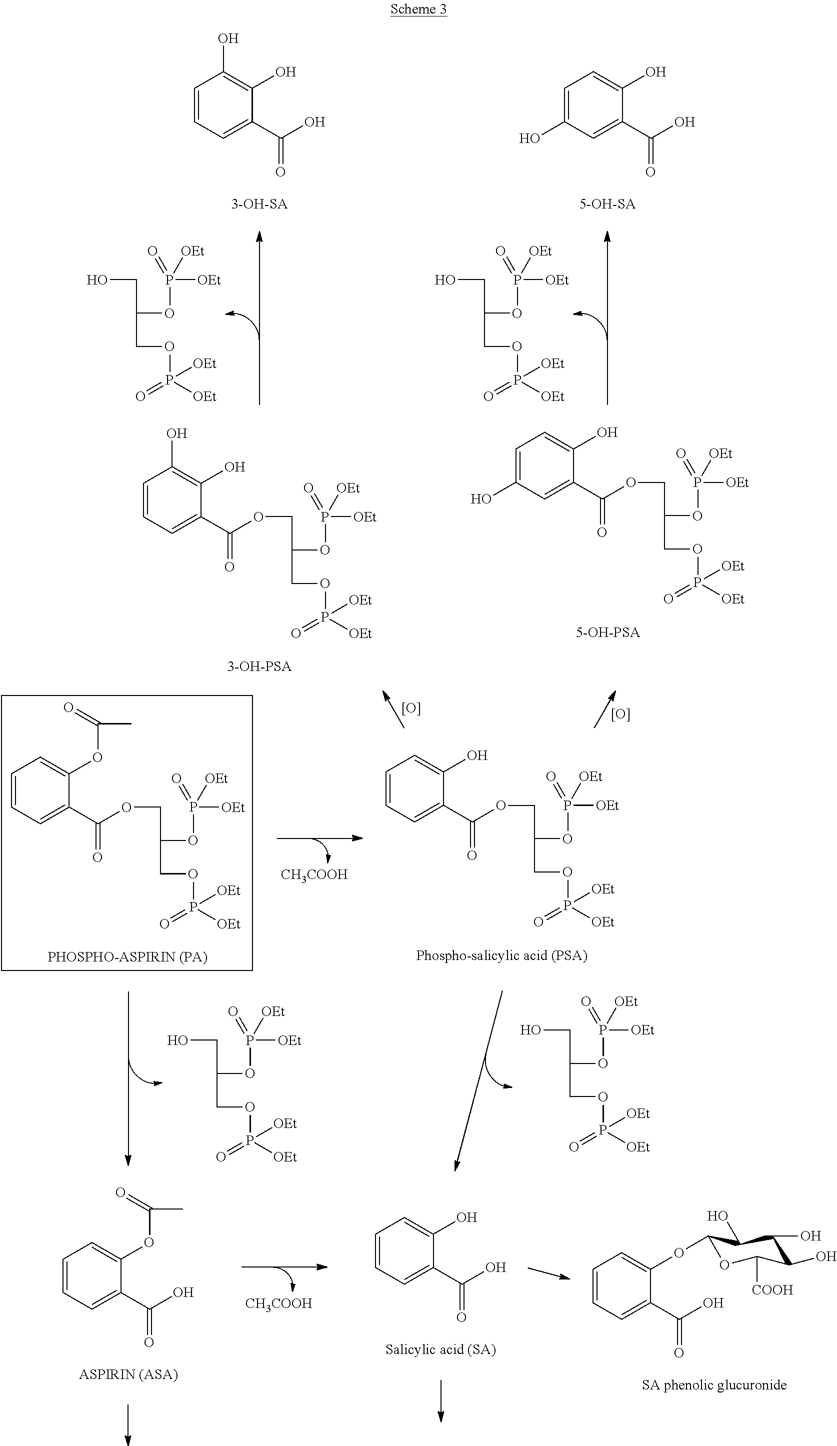

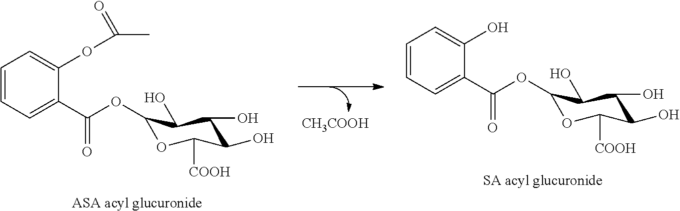

Subsequently, the metabolism of PA was further investigated.

Methods: The Metabolism of PSA by Human CYP Isoforms:

PSA was pre-incubated at 37° C. for 5 min with an NADPH-regenerating solution in 0.1 M potassium phosphate buffer (pH 7.4). The reaction was initiated by the addition of individual recombinant human CYP isoforms (25 pmol/ml) in a total volume of 1 ml and samples were maintained at 37° C. for various time periods. At each designated time-point, aliquots were extracted with acetonitrile, and subjected to HPLC analysis.

Results:

As shown in FIG. 3, three major metabolites of PA were detected in PA-treated liver microsomes by HPLC: phospho-salicylic acid [PSA], 3-OH-PSA and 5-OH-PSA. This result indicates that PA can be readily deacetylated at its ASA moiety to form PSA, which is oxidized to 3-OH-PSA and 5-OH-PSA. In contrast to PSA, conventional SA and ASA were not appreciably oxidized by liver microsomes under the same experimental conditions. Thus, a) only PSA can be oxidized to generate 3-OH-PSA and 5-OH-PSA (structures shown in Scheme 3 above), which are metabolites unique to PA vis-à-vis ASA or SA. Their generation represents regioselective oxidation; b) CYPs 2C19 and 2D6 catalyze appreciably this oxidation, with 1A2, 2C9 and 3A4 being minimally active; and c) ASA cannot be oxidized by any of the five CYPs tested, not even by whole liver microsomes.

These results indicate that PA can generate metabolites, which ASA cannot generate; and the production of these metabolites is catalyzed by a specific subset of CYP isoforms.

### Example 3

### Formation of Reactive Phospho-Aspirin (PA) Metabolites In Vivo

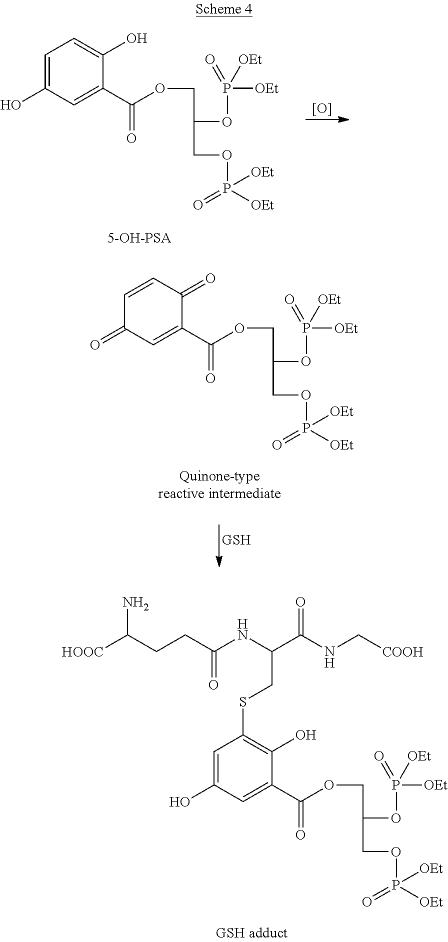

As shown in Scheme 4, 5-OH-PSA, having two hydroxyl groups, is dehydrogenated by CYPs to form a quinone-type highly reactive intermediate, which was trapped by GSH. Scheme 4 also shows the GSH adduct of a quinone-type reactive intermediate of PA, which was subsequently identified by LC-MS/MS analysis. Likewise, 3-OH-PSA leads to the analogous reactive intermediate, which was also trapped by GSH (data not shown). Quinones are highly redox-active molecules leading, among others, to reactive oxygen species (J. L. Bolton et al. Chem Res Toxicol. 2000, 13, 135-60), which, in turn, can induce cancer cell apoptosis (B. Rigas, Y. Br J Cancer. 2008, 98, 1157-60). Indeed, as recently shown, phospho-NSAIDs including PA, act by inducing oxidative stress selectively in cancer cells (Y. Sun et al. J Pharmacol Exp Ther. 2011, 338, 775-83).

These data explain a) the efficacy of PA BC prevention, as shown in Example 1 (PA produces the highly reactive quinone-type intermediates) and b) the lack of efficacy of ASA in the same study (ASA is unable to generate these reactive metabolites).

### Example 4

### Phospho-Farnesylthiosalicylic Acid (P-FTS, 35) Reduces Human Pancreatic Tumor Growth

Phospho-Farnesylthiosalicylic Acid (P-FTS, 35) Inhibits the Growth of Human Pancreatic Cancer Cells in Culture:

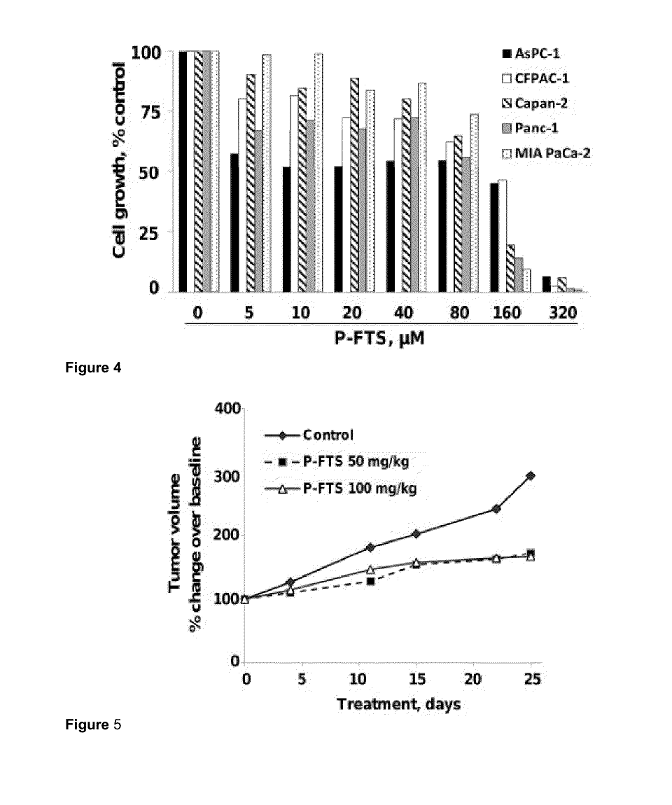

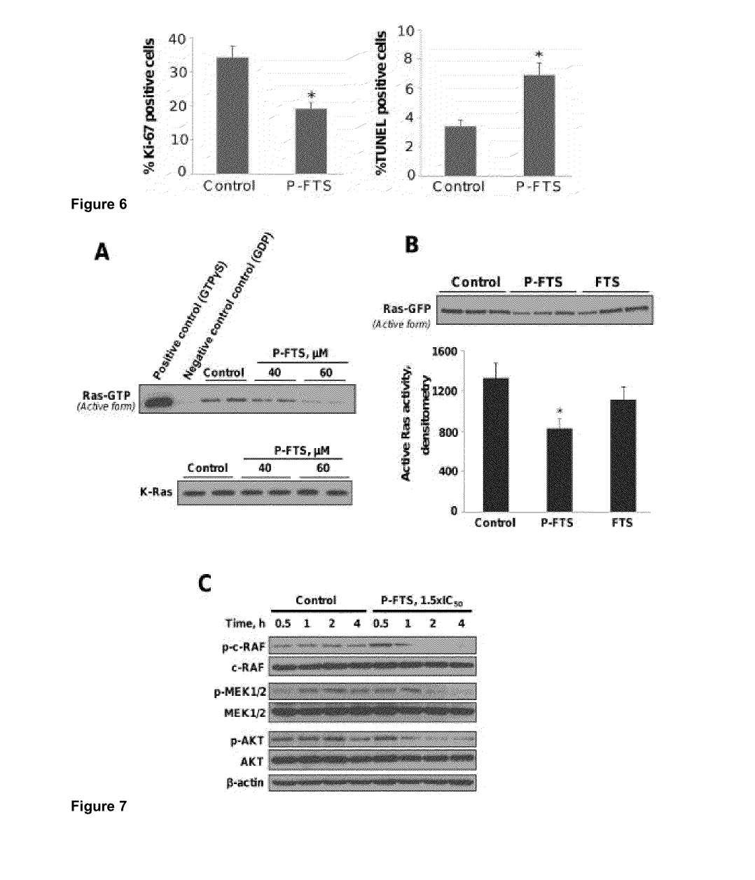

Cell growth was determined in the human pancreatic cancer cell lines AsPC-1, CFPAC-1, Capan-2, Panc-1 and MIA PaCa-2 after treatment with escalating concentrations of P-FTS 35 for 24 h. Results, expressed as % control, are show that P-FTS 35 decreases pancreatic cancer cell growth in a concentration-dependent manner in all cell lines (FIG. 4).

P-FTS Inhibits the Growth of Human Pancreatic Xenografts in Nude Mice;

The in vivo chemotherapeutic potential of P-FTS 35 was assessed using a pancreatic cancer xenograft model. MIA PaCa-2 cells were injected subcutaneously into the flank areas of nude mice. When palpable tumors were observed, the mice received P-FTS 35, 50 or 100 mg/kg/d by oral gavage in corn oil or just corn oil (control) for 25 days. On day 25 of treatment, P-FTS 35, 50 mg/kg/d, and P-FTS 35, 100 mg/kg/d reduced the tumor volume growth by 62% and 65%, respectively (p<0.05; FIG. 5).

### Safety of P-FTS in Mice

In the efficacy study described above, P-FTS 35 was well tolerated, with the mice showing no weight loss or other signs of toxicity. The toxicity of P-FTS 35 in mice was further examined. Groups of 6 week-old female BALB/c mice (5 mice/group) were given by oral gavage once a day for 3 weeks P-FTS 35: 0, 75, 150, 250 and 350 mg/kg. All P-FTS-treated animals showed no weight loss or other signs of toxicity. The (sub-chronic) maximum tolerated dose of P-FTS 35 (3-wk period of observation) was determined to be at least 350 mg/kg/d.

### P-FTS Inhibits Ras Activation and its Downstream Effectors ERK and AKT

In the following experiments it was examined whether the pancreatic cancer growth inhibitory effect of P-FTS 35 is associated with down regulation of Ras signaling. For this purpose, we used the Ras-GTP pull down assay. In Panc-1 cells, P-FTS 35 significantly inhibited active Ras (Ras-GTP) in a concentration-dependent manner, and this decrease was more pronounced that the one observed with the parent compound (FIG. 7). The inhibition of Ras by P-FTS 35 led to a significant time-dependent inhibition of the RAF/MEK/ERK and PI3K/AKT pathways, two downstream effectors of Ras (FIG. 7C).

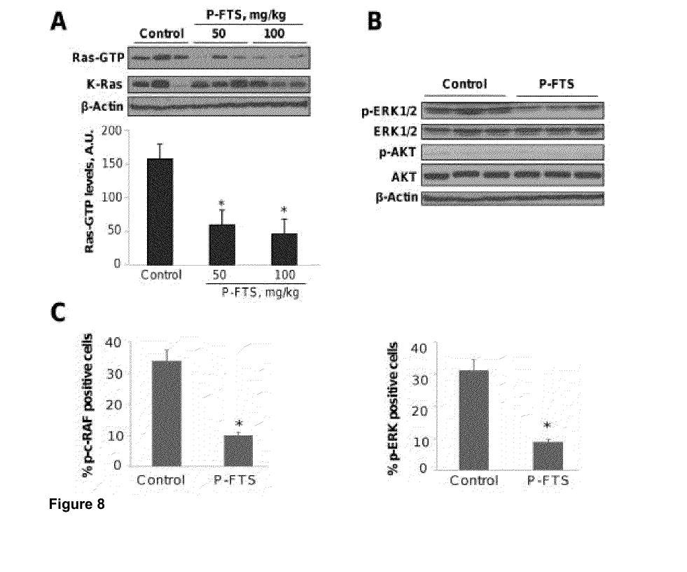

The inhibition of Ras by P-FTS 35 was further confirmed in vivo. The Ras-GTP pull down assay was used to test the capacity of P-FTS 35 to inhibit active Ras in fresh protein lysates from MIA PaCa-2 xenografts. The observed results are summarized in FIG. 8. Compared to controls, P-FTS 35, 50 and 100 mg/kg reduced RAS activation in xenografts by 62% and 70%, respectively (p<0.01, for both; FIG. 8A). The suppression of Ras was accompanied by inhibition of p-ERK and p-AKT, as determined by immunoblotting (FIG. 8B). Moreover, immunostaining of xenograft tissue sections revealed that P-FTS reduced the expression of p-c-RAF and p-ERK1/2 by 73% and 71%, respectively, compared to control (p<0.01; FIG. 8C).

In summary, the obtained in vivo results suggest, in agreement with the in vitro results, that Ras is a critical molecular target of P-FTS 35, likely accounting for its pancreatic cancer growth inhibitory effect.

P-FTS 35 Synergizes with Phospho-Valproic Acid (P-V, 64) to Enhance Human Pancreatic Cancer Growth Inhibition

The potential synergy between P-FTS 35 with phospho-valproic acid (P-V, 64), a novel STAT3 inhibitor was evaluated in BxPC-3 and MIA PaCa-2 cells. The structure of phospho-valproic acid 64 is shown below:

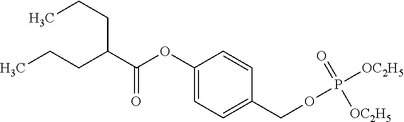 [SMILES: CCCC(CCC)C(=O)Oc1ccc(COP(=O)(OCC)OCC)cc1]

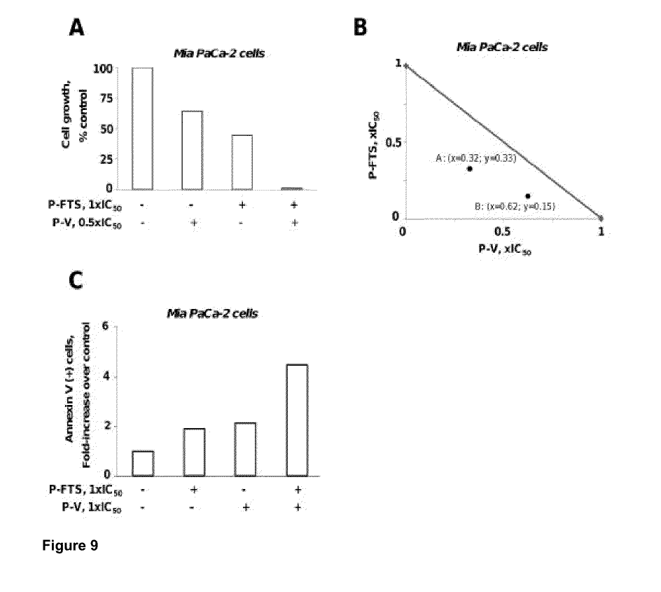

The results, summarized in FIG. 9, show P-FTS 35 and P-V 64 synergize to inhibit cell growth (FIGS. 9A and B) and induce apoptosis (FIG. 9C). These results suggest that P-V 64 is a useful combination partner of P-FTS 35 in the treatment of Ras mutated cancers.

1. A compound of Formula I 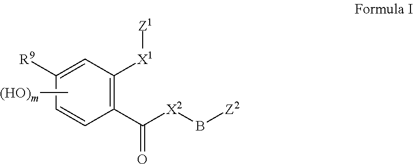
or an enantiomer, a diastereomer, a racemate, a tautomer, salt or hydrate thereof,
wherein
m—0 or 1;
X¹ and X² are independently selected from the group consisting of —O—, —S— and —NR¹—, R¹ being hydrogen or C₁₋₆-alkyl;
B is an optionally substituted aliphatic, heteroaliphatic, aromatic, heteroaromatic or alkylaryl substituent having 1 to 40 carbon atoms;
Z¹ is selected from the group consisting of hydrogen, farnesyl and a folic acid residue;
Z² is selected from the group consisting of 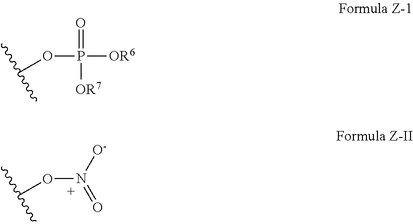
R⁶ being independently selected from hydrogen, C₁₋₁₀₀-alkyl and a polyethylene glycol residue;
R⁷ being independently selected from hydrogen, C₁₋₁₀₀-alkyl and a polyethylene glycol residue;
or B together with Z² forms a structure 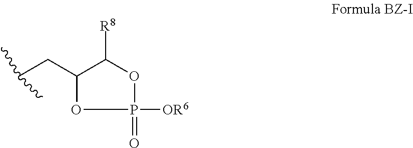
R⁶ being defined as above,
R⁸ being independently selected from hydrogen, an aliphatic substituent with 1 to 22 carbon atoms, more preferred C₁₋₆-alkyl and a polyethylene glycol residue and
R⁹ being hydrogen or trifluoromethyl.

2. The compound according to claim 1, wherein the folic acid residue is selected from the group consisting of

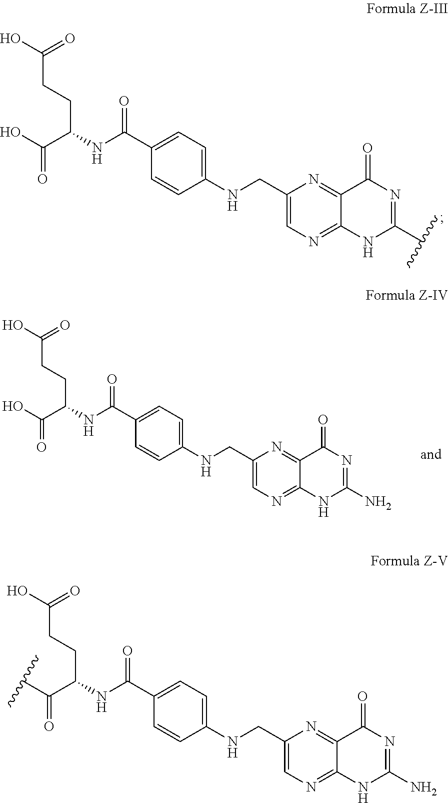 [SMILES: *c1nc(=O)c2nc(CNc3ccc(C(=O)N[C@@H](CCC(=O)O)C(=O)O)cc3)cnc2[nH]1 ; Nc1nc(=O)c2nc(CNc3ccc(C(=O)N[C@@H](CCC(=O)O)C(=O)O)cc3)cnc2[nH]1 ; *C(=O)[C@H](CCC(=O)O)NC(=O)c1ccc(NCc2cnc3[nH]c(N)nc(=O)c3n2)cc1]

3. The compound according to claim 1, wherein Z¹ is hydrogen.

4. The compound according to claim 1, wherein Z¹ is farnesyl.

5. A compound of Formula II 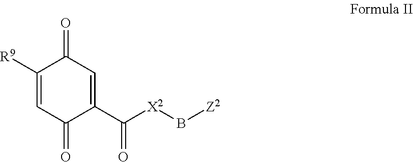
or an enantiomer, a disatereomer, a racemate, a tautomer, salt or hydrate thereof, wherein X², B and Z² are as defined above.

6. The compound according to any of claims 1 to 5, wherein
B is selected from the group consisting of 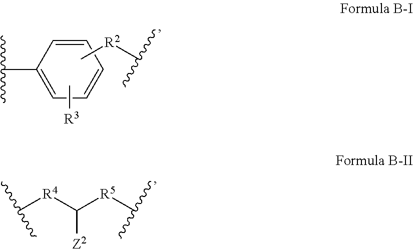
and an aliphatic substituent with 1 to 40 carbon atoms,
R², R⁴ and R⁵ being the same or different C₁₋₃-alkylene,
R³ being hydrogen, C₁₋₆-alkyl, halogenated C₁₋₆-alkyl, C₁₋₆-alkoxy, halogenated C₁₋₆-alkoxy, —C(O)—C₁₋₆-alkyl, —C(O)O—C₁₋₆-alkyl, —OC(O)—C₁₋₆-alkyl, —C(O)NH₂, —C(O)NH—C₁₋₆-alkyl, —S(O)—C₁₋₆-alkyl, —S(O)₂—C₁₋₆-alkyl, —S(O)2NH—C₁₋₆-alkyl, cyano, halo or hydroxy.

7. The compound according to any of claims 1 to 6, wherein X² is —NR¹—, R¹ is hydrogen;
B is selected from the group consisting of 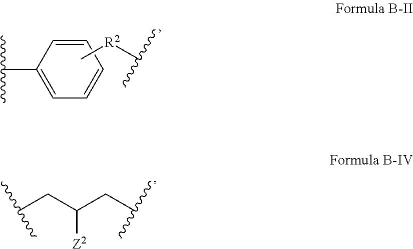
C₁₋₆-alkylene, C₂₋₆-alkenylene and C₂₋₆-alkynylene;
Z² is represented by Formula Z-I,
R⁶ being independently selected from hydrogen, C₁₋₃-alkyl and (OCH₂CH₂)—OCH₃,
R⁷ being independently selected from C₁₋₃-alkyl and (OCH₂CH₂)—OCH₃,
whereby n is from 40 to 50.

8. The compound according to any of claims 1 to 7, wherein X¹ is —NR¹—, R¹ is hydrogen;
B is selected from the group consisting of C₁₋₄-alkylene and 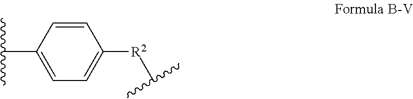
R² being methylene or ethylene; and
Z² is represented by Formula Z-I, R⁶ and R⁷ being identical C₁₋₃-alkyl substituents.

9. The compound according to any one of claims 1 to 8, wherein X² is —NR¹—, R¹ is hydrogen; B is —(CH₂)₄—; and Z² is represented by Formula Z-1, R⁶ and R⁷ being identical C₁₋₃-alkyl substituents.

10. The compound according to any one of claims 1 to 9, wherein X² is —NH—; B is —(CH₂)₄—; and Z² is represented by Formula Z-1, R⁶ and R⁷ being identical C₁₋₃-alkyl substituents.

11. A compound according to any one of claims 1 to 10 for use in the treatment and/or prevention of disorders selected from the group consisting of inflammation, pain, fever, cancer and precancerous conditions.

12. The compound according to claim 11, wherein the disorder is a cancer with a mutant Ras gene such as pancreatic cancer.

13. A pharmaceutical composition comprising the compound according to any of claims 1 to 10 for use in the treatment and/or prevention of disorders selected from the group consisting of inflammation, pain, fever, cancer or precancerous conditions.

14. The pharmaceutical composition according to claim 13, wherein said pharmaceutical composition is formulated in the form of nanoparticles.

15. The pharmaceutical composition according to claim 13 or 14, wherein said pharmaceutical composition further comprises one or more additional compounds having anti-cancer activity.

16. A compound selected from the group consisting of compounds 1 to 169.

17-23. (canceled)

24. A pharmaceutical composition comprising compound of claim 1.
